(19) United States
(12) Patent Application Publication
(10) Pub. No.: US 2005/0208499 A1
(43) Pub. Date: Sep. 22, 2005

(54) Markers for diagnosing and treating breast and ovarian cancer

(76) Inventors: Jonathan M. Graff, Rockwall, TX (US); Deborah A. Ferguson, Dallas, TX (US); Qun Zang, Dallas, TX (US); Jeffrey A. Spencer, Grand Prairie, TX (US)

Correspondence Address: M. Michelle Muller;VINSON & ELKINS LLP, 2300 First City Tower, 1001 Fannin, Houston, TX, 77002-6760, US

(21) Appl. No.: 10/771,620

(22) Filed: Feb. 4, 2004

(51) Int. Cl. C12Q 1/68

(52) U.S. Cl. 435/6

(57) ABSTRACT

This invention relates to methods, kits and compositions for diagnosing, assessing the prognosis or monitoring the progression of breast or ovarian cancer in a subject. In particular, the invention discloses methods for detecting changes in the expression pattern of CXCL9 and FLJ20174 nucleic acids and gene products as markers for breast and ovarian cancer. Also disclosed are methods and kits for screening compounds for the ability to act as effectors of breast and ovarian cancer and therapeutic agents for the treatment of breast or ovarian cancer that specifically bind CXCL9 or FLJ20174 nucleic acids or gene products.

### STATEMENT REGARDING FEDERALLY SPONSORED RESEARCH or DEVELOPMENT

This work was funded by the Department of Defense, Grant or Contract #: DAMD170210591.

### BACKGROUND OF THE INVENTION

1. Field of the Invention

The present disclosure is related to materials and methods for diagnosing and treating breast and ovarian cancer in a subject, and determining the prognosis of the subject by determining whether the expression of chemokine (CXC motif) ligand 9 (CXCL9) or FLJ20174 is up-regulated in a biological sample.

2. Description of Related Art

Breast cancer is a major cause of illness and death in women world-wide, and morbidity and mortality due to this disease remain unacceptably high. In the United States, there are over 200,000 new cases of breast cancer, and almost 40,000 deaths per year result from this disease. Many prognostic indicators aid in the evaluation of invasive breast cancer such as the presence or absence of lymph node metastasis, tumor size, histologic type, tumor grade (differentiation reflected in extent of gland formation), nuclear grade (extent of nuclear alteration and frequency of mitosis), DNA content (ploidy), and hormone receptor status. This heterogeneity in prognostic indicators reflects the major challenges health care providers still face in the diagnosis and treatment of breast cancer. Breast cancers also appear to be genetically and/or morphologically heterogeneous, with multiple mechanisms responsible for the ultimate development and risk of the breast carcinoma. In addition, screening methods are needed to identify early stage transition of normal cells towards cancerous cells before the subsequent development of invasive and metastatic cancer.

Currently, there are few selective markers for breast cancer. Her-2/neu, an example of one such marker, is an oncogene that was identified in an amplified form in a human breast cancer cell line (King et al., Science 229:974-76, 1985). This marker has limited utility though because it is only expressed in approximately 20-30% of the breast cancer samples tested. Tan et al., WO 03/070979, discloses a method wherein a plurality of markers including the CXCL9 marker are used to determine the presence of breast cancer, to determine the type of breast cancer that is present, or both. The method however requires testing of numerous markers in a particular sub-set of patients in order to reach a determination about the presence or sub-type of breast cancer cells.

Thus, there is a great need for selective molecular markers that are differentially expressed in a higher percentage of breast cancers, and can be used in a diagnostic assay to determine the presence or risk of breast cancer in a subject. Specifically, there is a need for the identification of a marker that is differentially expressed in greater than 50% of breast cancers relative to non-cancerous cells. Ideally, the expression level of this marker will also allow health care providers to risk-stratify breast cancer subjects into low-risk and high-risk groups, particularly in terms of the risk or recurrence of the disease after treatment.

Similarly, ovarian cancer is a significant health problem for women in the United States and throughout the world. According to the American Cancer Society, ovarian cancer ranks as the fifth leading cause of death from cancer among women. Although advances have been made in detection and therapy of this cancer, no vaccine or other universally successful method for prevention or treatment is currently available. In fact, the death rate for this disease has not changed much in the last 50 years.

Management of ovarian cancer currently relies on a combination of early diagnosis and aggressive treatment, which may include one or more of a variety of treatments such as surgery, radiotherapy, chemotherapy and hormone therapy. Chemotherapeutic agents including cisplatin, carboplatin, paclitaxel, topotecan, pegylated liposomal doxorubicin, gemcitabine and oral etoposide have been used to either treat the disease or prolong survival. However, despite the fact that early diagnosis is critical to survival, almost 70% of women with the common epithelial ovarian cancer are not diagnosed until the cancer has reached an advanced stage, significantly reducing the chances of survival. Both diagnosis and selection of a course of treatment for a particular cancer is often based on a variety of prognostic parameters, including an analysis of specific tumor markers, but the use of established markers often leads to results that are uninformative or difficult to interpret. Thus, there is a great need for selective molecular markers that are differentially expressed in a higher percentage of ovarian cancers, and can be used in diagnostic assay to determine the presence or risk of a subject for ovarian cancer and to suggest appropriate treatment protocols.

The nucleic acid sequence of CXCL9 has been identified as encoding chemokine (C—X—C motif) ligand 9. The CXCL9 protein is a secreted protein belonging to a family of cytokines believed to be involved in cell-specific chemotaxis, mediation of cell growth and the inflammatory response. Molecules in this family are typically cationic proteins of 70 to 100 amino acids that share four conserved cysteine residues involved in two disulfide bonds. These proteins can be sorted into two groups based on the spacing of the two amino-terminal cysteines. In the first group, the two cysteines are separated by a single residue (C—X—C motif), while in the second group the cysteines are adjacent (C—C motif). The CXCL9 gene product exhibits the C—X—C motif. The molecule has been shown to have anti-tumor activity in vivo. (Ruehlmann et al., “MIG (CXCL9) chemokine gene therapy combines with antibody-cytokine fusion protein to suppress growth and dissemination of murine colon carcinoma,” Cancer Res. 61(23):8498-503, 2001; Addison et al., “The CXC chemokine, monokine induced by interferon-gamma, inhibits non-small cell lung carcinoma tumor growth and metastasis,” Human Gene Ther. 11(2):247-61, 2000; Sgadari et al., “Mig, the monokine induced by interferon-gamma, promotes tumor necrosis in vivo,” Blood 89(8):2635-43, 1997; Dorsey et al., “Immunotherapy and Interleukin-10 Depends on the CXC Chemokines Inducible Protein-10 and Monokine Induced by IFN-γ,” Cancer Research 62:2606-10, 2002.) However, despite its anti-tumor activity, aberrant expression of CXCL9 has not previously been identified as a marker for ovarian or breast cancer.

FLJ20174 is a gene sequence of unknown function encoding a hypothetical protein. FLJ20174 is homologous to the Caenorhabditis elegans SID-1 gene which is believed to be involved in systemic RNA interference (RNAi) (Feinberg et al., “Transport of dsRNA into cells by the transmembrane protein SID-1,” Science 301:1545-7, 2003; Winston et al., “Systemic RNAi in C. elegans requires the putative transmembrane protein SID-1”, Science 295:2456-9, 2002.) A homolog of FLJ20174, NOV2, was identified by Guo et al., WO 02/46409. NOV2 was identified as having utility as a therapeutic agent for cancer therapy and as a diagnostic. Afar et al., WO 03/042661 identifies FLJ20174 as a gene that is up-regulated or down-regulated in specific diseases. Neither Guo et al. nor Afar et al., however, identify FLJ20174 as having specific utility as a marker for ovarian or breast cancer.

### BRIEF SUMMARY OF THE INVENTION

The present invention provides compositions, methods and kits for diagnosing breast cancer in a subject. In particular, the present invention provides a method of diagnosing breast cancer in a subject, the method comprising comparing the expression pattern of CXCL9 or FLJ20174 in a sample from a subject with the expression pattern of CXCL9 or FLJ20174 in samples from one or more non-cancerous tissues. A sample from a non-cancerous tissue can be obtained from the same type of tissue as the sample from the subject or can be obtained from another type of tissue. Similarly the sample from the subject and the one or more samples from a non-cancerous tissue can be obtained from the same subject or from different subjects. In preferred embodiments, the CXCL9 or FLJ20174 expression pattern in the subject is an up-regulation of the expression level of CXCL9 or FLJ20174 in the sample from the subject with respect to the one or more samples from non-cancerous tissues. The present invention also provides a kit for the diagnosis of breast or ovarian cancer, the kit comprising reagents for assessing the expression pattern of CXCL9 or FLJ20174 in a biological sample.

A sample of the present invention can comprise cells obtained from a subject, including cells obtained from breast or ovarian tissue. In other embodiments, the biological sample can comprise serum, nipple aspirate, ductal fluid or other tissue, cells or fluids obtained in a biopsy of the subject. The subject may be living or dead at the time of the examination of the sample.

The methods of the present invention include methods of determining the expression pattern of CXCL9 or FLJ20174 by detecting the presence in the sample of a nucleic acid comprising 20, 25, 30, 35, 40, 45, 50, 75, 100, 200 contiguous nucleotides of SEQ ID NO: 1, SEQ ID NO:3 or SEQ ID NO:4. SEQ ID NO: 1, SEQ ID NO:3 and SEQ ID NO:4 are cDNA sequences, but throughout the specification the methods and compositions of the present invention relating to SEQ ID NO: 1, SEQ ID NO:3 and SEQ ID NO:4 apply equally to any corresponding nucleic acid sequence, including the corresponding DNA or RNA sequences, or portions thereof. Particular embodiments of this method include methods wherein the expression pattern of CXCL9 or FLJ20174 in the sample is assessed by detecting the presence in the sample of a polynucleotide encoded by SEQ ID NO:1, SEQ ID NO:3 or SEQ ID NO:4. or a portion of said transcribed polynucleotide. In preferred embodiments, the transcribed polynucleotide is an mRNA or hnRNA or a cDNA derived therefrom. In further preferred embodiments, the step of detecting the transcribed polynucleotide further comprises amplifying the transcribed polynucleotide. In an alternate embodiment, the expression pattern of CXCL9 or FLJ20174 in the samples is assessed by detecting the presence in the samples of a transcribed polynucleotide which specifically binds with SEQ ID NO:1, SEQ ID NO:3 or SEQ ID NO:4 or specifically binds with a portion of said transcribed polynucleotide, under stringent hybridization conditions.

The present invention also provides methods of detecting the expression pattern of CXCL9 or FLJ20174 wherein the expression pattern of CXCL9 or FLJ20174 is determined by detecting the presence in the samples of a protein, polypeptide or protein fragment of SEQ ID NO:2, SEQ ID NO:5 or SEQ ID NO:6. Said protein, polypeptide or protein fragment can comprise 8, 9, 10, 11, 12, 13, 14, 15, 16, 17, 18, 19, 20, 25, 30, 40, 50 or more contiguous amino acids of SEQ ID NO:2, SEQ ID NO:5 or SEQ ID NO:6. In certain embodiments, the presence of said protein, polypeptide or protein fragment is detected using a reagent which specifically binds with said protein, polypeptide or protein fragment. In particular embodiments, the reagent can be selected from the group consisting of an antibody, an antibody derivative, and an antibody fragment.

The present invention further provides a method of assessing the prognosis of a breast cancer subject, the method comprising comparing the expression pattern of CXCL9 or FLJ20174 in a biological sample with the expression pattern of CXCL9 or FLJ20174 in samples from one or more subjects suffering from a known type of breast cancer and determining the prognosis based on the comparison. In preferred embodiments, the method is one wherein the expression pattern is assessed by determining the level of expression. In alternate embodiments, the expression pattern is assessed by comparing expression patterns of CXCL9 or FLJ20174 in different tissue samples from the same subject. In other embodiments, the expression pattern is assessed by comparing the level of post-translational modification of CXCL9 or FLJ20174.

Other methods of the present invention include a method for monitoring the progression of breast or ovarian cancer in a subject, the method comprising: a) determining in a biological sample at a first point in time the expression pattern of CXCL9; b) repeating step a) at a subsequent point in time; and c) comparing the expression pattern of the CXCL9 determined in steps a) and b), and therefrom monitoring the progression of breast or ovarian cancer. In preferred embodiments, between the first point in time and the subsequent point in time, the subject has undergone surgery to remove breast or ovarian tissue.

The present invention also provides methods, kits and compositions for assessing the potential of one or more test compounds to act as an effector of breast or ovarian cancer in a subject. The method comprises the steps of: (a) determining the expression pattern of CXCL9 or FLJ20174 in a first cancer tissue sample from a subject wherein the tissue exhibits an abnormal CXCL9 or FLJ20174 expression pattern; (b) exposing one or more second cancer tissue samples from the same subject to one or more test compounds; (c) determining the expression pattern of CXCL9 or FLJ20174 in the one or more tissue samples that have been exposed to one or more test compounds; and (d) comparing the expression pattern of CXCL9 or FLJ20174 in the first and second tissue samples, wherein a change in the expression pattern of CXCL9 or FLJ20174 in the second tissue sample versus the first tissue sample indicates that the test compound is an effector of breast or ovarian cancer in a subject. This method can be used to assess test compounds that are therapeutic agents for breast and ovarian cancer. This method can also be used to assess the ovarian or breast cell carcinogenic potential of a test compound.

The present invention therefore provides methods and kits for selecting a therapeutic agent for inhibiting breast or ovarian cancer in a subject, the method comprising: a) obtaining a sample comprising cancer cells from the subject; b) separately exposing aliquots of the sample to one or more test compounds; c) comparing the expression pattern of CXCL9 or FLJ20174 in each of the aliquots; and d) selecting one or more of the test compounds which alter the expression pattern of CXCL9 or FLJ20174 in a desirable manner. In preferred embodiments, the method further comprises the step of administering the selected one or more test compounds to a subject as a treatment for breast or ovarian cancer. Further, the present invention provides a kit for assessing the suitability of one or more test compounds for inhibiting breast cancer in a subject, the kit comprising: a) one or more test compounds; and b) a reagent for assessing the expression pattern of CXCL9 or FLJ20174. In certain embodiments the methods or kits comprise a reagent which specifically binds with a transcribed polynucleotide of SEQ ID NO:1, SEQ ID NO:3 or SEQ ID NO:4, or to a portion of said transcribed polynucleotide. In alternate embodiments, the methods or kits comprise a reagent which specifically binds to a protein, polypeptide or protein fragment of SEQ ID NO:2, SEQ ID NO:5 or SEQ ID NO:6.

Finally, the present invention provides therapeutic agents for the treatment of breast or ovarian cancer comprising an agent that specifically binds a CXCL9 or FLJ20174 nucleic acid or gene product. The therapeutic agent of the present invention can be an agent that specifically binds to a protein, polypeptide or protein fragment of SEQ ID NO:2, SEQ ID NO:5 or SEQ ID NO:6 including, but not limited to, an antibody, antibody derivative, and an antibody fragment. Alternatively, the therapeutic agent of the present invention can be an agent that specifically binds with a polynucleotide of SEQ ID NO: 1, SEQ ID NO:3 or SEQ ID NO:4 including, but not limited to antisense nucleic acids and RNA interference oligonucleotides.

### BRIEF DESCRIPTION OF THE SEVERAL VIEWS OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Tissue panel assessing levels of CXCL9 gene expression in a variety of normal and cancerous tissues by means of RT-PCR.

FIG. 2. Breast cancer panel assessing levels of CXCL9 gene expression in normal breast tissue and in ductal carcinomas by means of RT-PCR.

FIG. 3. Ovarian cancer panel assessing levels of CXCL9 gene expression in normal ovarian tissues and in ovarian adenocarcinomas by means of RT-PCR.

FIG. 4. Tissue panel assessing levels of FLJ20174 gene expression in a variety of normal and cancerous tissues by means of RT-PCR.

FIG. 5. Tissue panel assessing levels of FLJ20174 gene expression in a variety of normal and cancerous tissues by means of RT-PCR.

FIG. 6. Tissue panel assessing levels of FLJ20174 gene expression in a variety of normal and cancerous tissues by means of RT-PCR. The upper half of the figure shows the expression patterns of both the wild-type and splice variant forms of FLJ20174.

FIG. 7. Breast cancer panel assessing levels of FLJ20174 gene expression in normal breast tissue and in ductal carcinomas by means of RT-PCR.

FIG. 8. Breast cancer panel assessing levels of FLJ20174 gene expression in normal breast tissue and in ductal carcinomas by means of RT-PCR. The upper half of the figure shows the expression of both the wild-type and splice variant forms of FLJ20174.

FIG. 9. Ovarian cancer panel assessing levels of FLJ20174 gene expression in normal ovarian tissues and in ovarian adenocarcinomas by means of RT-PCR.

FIG. 10. Ovarian cancer panel assessing levels of FLJ20174 gene expression in normal ovarian tissues and in ovarian adenocarcinomas by means of RT-PCR. The upper half of the figure shows the expression of both the wild-type and splice variant forms of FLJ20174.

FIG. 11. Quantitative RT-PCR of CXCL9 expression in normal tissues, breast cancers and ovarian cancers.

FIG. 12. Quantitative RT-PCR of FLJ20174 expression in normal tissues, breast cancers, and ovarian cancers.

### DETAILED DESCRIPTION OF THE INVENTION

The present disclosure relates to methods of detecting breast or ovarian cancer in a subject, or determining the prognosis of a subject having cancer of both by determining whether CXCL9 or FLJ20174 is up-regulated or differentially expressed as compared to normal tissues in a biological sample, such as a biopsy from the subject. The upregulated or otherwise altered expression of CXCL9 or FLJ20174 in a biological sample relative to a normal control indicates the presence of cancer, and may also indicate an increased or decreased chance of long-term survival for the subject. In preferred embodiments, the subject is a human cancer patient.

As used herein, the term “CXCL9 gene” or “CXCL9 nucleic acid sequence” and the like refer to the nucleic acid disclosed as SEQ ID NO:1 and homologs, mutations or variants of that sequence found in a subject. The “expression pattern” or “level of expression” of CXCL9 refers to the characteristics of mRNA or hnRNA transcripts transcribed from the CXCL9 gene and to the characteristics of the gene products translated from those mRNA transcripts. As used herein, the “CXCL9 gene product” is the amino acid sequence of SEQ ID NO:2 variants or fragments of that sequence. Detectable alterations in the expression pattern of the gene product of CXCL9 can include changes in the level of expression of the protein or changes in the gene products normal characteristics, such as its molecular weight, amino acid composition or post-translational modification and the like.

FLJ20174 is expressed in human tissue in two isoforms. One isoform is designated herein as the “wild-type” form and, while not bound to theory, is believed to correspond to the GenBank accession number NM_{—}017699 nucleic acid sequence (SEQ ID NO: and GenBank accession number NP_{—}060169 amino acid sequence. The second isoform is designated herein as the “splice-variant” and, not to be bound by theory, is believed to be an mRNA corresponding to the FLJ20174 gene sequence, except that the mRNA transcript is missing predicted exon 15 of the NM_{—}017699 sequence. Due to a frame shift, the loss of the exon sequence in the transcript results in a truncated polypeptide. Both isoform sequences further show homology to GenBank accession numbers AC112128 and AC055740.

As used herein, the term “FLJ20174 gene” or “FLJ20174 nucleic acid sequence” and the like refers to the nucleic acid sequence disclosed as SEQ ID NO:3 and SEQ ID NO:4 and homologs, mutations or variants of those sequences found in a subject. The term therefore includes both the “wild-type” and “splice-variant” isoforms of FLJ20174 discussed above. The “expression pattern” or “level of expression” of FLJ20174 refers to the characteristics of mRNA or hnRNA transcripts transcribed from the FLJ20174 gene and to the characteristics of the gene products translated from those mRNA transcripts. As used herein, the “FLJ20174 gene product” is the amino acid sequence of SEQ ID NO:5 and SEQ ID NO:6, and variants or fragments of those sequences. Detectable alterations in the expression pattern of the gene product of FLJ20174 can include changes in the level of expression of the protein or changes in the gene products normal characteristics, such as its molecular weight, amino acid composition or post-translational modification and the like.

Bioassays for Determining the Amount of CXCL9 or FLJ20174 Present in a Biological Sample:

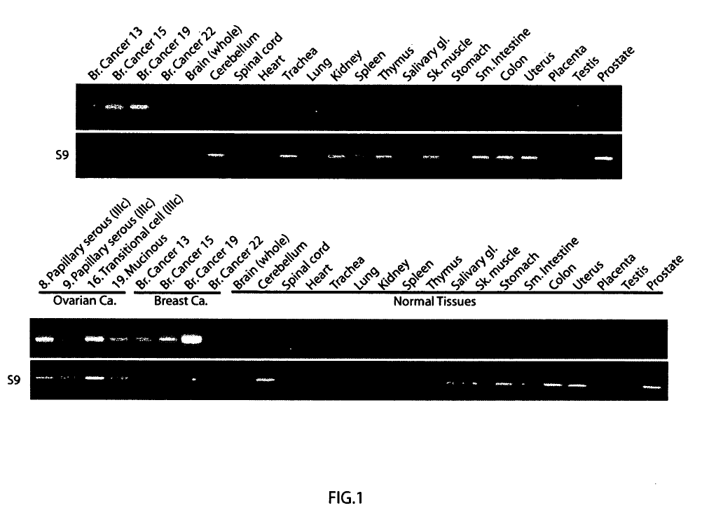

As shown in FIGS. 1-10, high expression levels of CXCL9 and FLJ20174 mRNA have been detected in breast cancer and ovarian cancer samples, while normal tissues show minimal or undetectable expression of CXCL9 or FLJ20174. Expression of CXCL9 or FLJ20174 may be upregulated in a cancerous cell by several different mechanisms including, but not limited to the upregulation of the gene encoding CXCL9 or FLJ20174, the duplication of the gene in the cell, so that multiple copies of the gene are being expressed in the cell, alterations in the mRNA transcript produced by the gene resulting in increased stability of the mRNA, or alterations in post-translational modification of the expressed protein resulting in increased stability of the protein.

As used herein, a “sample” or “biological sample” includes but is not limited to biological fluid, a tissue extract or sample, freshly harvested cells, lysates of cells, or a biopsy from a subject, such as blood, plasma, serum, and urine, breast fluid or any tissue or cell taken from surgical biopsies or otherwise isolated from a subject. Preferably the subject is human. As used herein, a “biopsy” means any body fluids, tissues or cells which may contain CXCL9 or FLJ20174 nucleic acids or gene products, which have been removed from a subject suspected of having breast or ovarian cancer. As used herein, a sample or biopsy from a cancerous or non-cancerous tissue is a sample or biopsy as described above which contains CXCL9 or FLJ20174 nucleic acids or gene products obtained from cancerous or non-cancerous cells.

Expression of CXCL9 mRNA is upregulated at least 2-fold in breast cancer tissues versus its expression level in normal tissues. Expression of CXCL9 mRNA can be even further elevated in breast cancer tissues, including increased expression of at least 5-fold, at least 10-fold, at least 20-fold, at least 30-fold, at least 40-fold, at least 50-fold, at least 100-fold or even at least 200-fold versus the expression level in non-cancerous breast tissue. Expression of CXCL9 mRNA can be elevated in ovarian cancer tissues at least 2-fold, at least 3-fold, at least 4-fold, at least 5-fold, at least 6-fold, at least 7-fold, at least 8-fold, or at least 10-fold. Expression of FLJ20174 mRNA can be elevated in breast or ovarian cancer tissues at least 1.4-fold, 2-fold, 3-fold, 4-fold, 5-fold, 8-fold or at least 10-fold versus non-cancerous breast or ovarian tissue, respectively. Similar levels of increased expression can be observed for CXCL9 or FLJ20174 gene products in breast and ovarian cancer versus the expression level of those gene products in non-cancerous breast or ovarian tissue, respectively.

One method of obtaining body fluids for testing is where the fluid is obtained from the breast, e.g., by nipple aspiration of the milk ducts or by ductal lavage of at least one breast milk duct. When fluid is collected by nipple aspiration, or by ductal lavage, the fluid can be collected from a single duct or from more than one duct. For example, the duct and the collection tube can be marked so that the analysis of the fluid is traceable to one or more specific ducts.

By the procedure of ductal lavage, ductal epithelial cells that line the walls of the ductal lumen are washed out of the duct. Methods of obtaining ductal fluid by means of ductal lavage are well-known in the art. For example, lavage or wash fluid can be infused into the duct, and the lavage fluid mixed with ductal fluid is collected. In some cases suction can be applied to the tool accessing the ductal lumen in order to retrieve a maximum amount of cells and/or fluid. Lavage or wash fluid can be infused into the duct, and collected. Alternatively, ductal fluid can be retrieved by a medical tool, e.g., a catheter or a cannula placed into the duct to infuse wash fluid to retrieve a mixture of wash and ductal fluids. The fluid from the breast duct can contain ductal epithelial cells, including cells of a stage considered to be precancerous or cancerous.

Nipple aspiration of breast ductal fluid is achieved by using vacuum pressure. Methods of obtaining nipple aspirates are well-known in the art. For example, nipple aspirate fluid can be actively retrieved as described in Goodson W H & King E B, Chapter 4: Discharges and Secretions of the Nipple, The Breast: Comprehensive Management of Benign and Malignant Diseases (1998) 2nd Ed. vol. 2, Bland & Kirby eds. W. B. Saunders Co, Philadelphia, Pa. pp. 51-74; Wrensch et al., (1992) American Journal of Epidemiology. 135(2):130-41; and Sauter et al., (1997) British Journal of Cancer. 76(4):494-501. Also fluid secretions from the nipple can be collected as they spontaneously appear on the nipple surface.

The ductal fluid or breast tissue may be analyzed in situ, i.e., inside the breast and inside the breast duct, e.g., where a particular marker can be introduced into the duct and can be identified from within the breast. In situ testing is considered a noninvasive means of examining the ductal epithelial cells or breast tissue. Ductal epithelial cells that are examined by the method of the invention can be examined in situ, or after the ductal epithelial cells have been removed from the breast of the patient by ductal lavage, nipple aspiration or other means. Methods of in situ analysis can include use of such molecular biology tools, methods, and materials as described in e.g., U.S. Pat. Nos. 5,169,774, 5,720,937, 5,677,171, 5,720,954, 5,725,856, 5,770,195, and 5,772,997. Markers to breast cancer and breast precancer described herein can be used for an in situ analysis of the breast duct.

The amount of CXCL9 or FLJ20174 nucleic acid or gene product present in a biological sample from a subject can be detected by measuring the expression level of the protein, the mRNA encoding CXCL9 or FLJ20174, or the copy number of the CXCL9 or FLJ20174 gene. The present disclosure provides methods for determining the amount of CXCL9 or FLJ20174 marker in a biological sample by contacting the biological sample with a substance that binds or detects the DNA, mRNA, or protein of the marker under conditions such that a complex between the marker and the substance is formed and can be detected. Preferably, the approximate amount of complex present in the sample can be calculated, which is indicative of the quantity of marker present in the sample.

In addition to determining the level of expression of CXCL9 or FLJ20174, the present invention also provides methods for assessing other characteristics of the expression pattern of these genes and gene products. In the case of the mRNA transcript of the CXCL9 or FLJ20174 genes, it is possible to detect and quantify the presence of splice variants or other modifications of the mRNA transcript. In the case of the gene product, it is possible to detect variations in the post-translational modifications of the proteins including, but not limited to, modifications such as phosphorylation and glycosylation.

Assessments of the expression pattern of CXCL9 and FLJ20174 may also be of differential levels of expression in various tissues, including comparisons of the expression patterns between healthy tissue and diseased tissue, including cancerous tissue.

The present invention therefore provides compositions, kits and methods for the detection of breast or ovarian cancer in a subject. The compositions, kits, and methods of the invention are further useful for characterizing the stage, grade, histological type, and benign or malignant nature of breast or ovarian cancer in a subject.

Expression of a marker gene of the invention may be assessed by any of a wide variety of well known methods for detecting expression of a transcribed RNA or encoded protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods. In situ hybridization (ISH) and immunohistochemistry (IHC) methods are preferred. Many of these methods involve the specific binding of a reagent to the nucleic acid or gene product being detected. As used herein, a reagent that “specifically binds” will preferentially bind a specific nucleic acid or gene product, or portion thereof, over any other nucleic acid or gene product found in a sample.

Detection of CXCL9 or FLJ20174 Nucleic Acid Molecules:

A variety of methods can be employed to screen for the presence of, or detect and/or assay levels of, CXCL9 or FLJ20174 nucleic acid sequences in a biological sample as compared to normal controls. CXCL9 or FLJ20174 genes can be detected by utilizing a number of techniques. Nucleic acid from any biological sample can be used as the starting point for such assay techniques, and may be isolated according to standard nucleic acid preparation procedures that are well known to those of skill in the art. CXCL9 or FLJ20174 nucleic acid sequences may be used in hybridization or amplification assays of biological samples to detect abnormal up-regulation of CXCL9 or FLJ20174, including but not limited to, Southern analyses, Northern analyses, single-stranded conformational polymorphism analyses (SSCP), PCR, and RT-PCR analyses. Such analyses may reveal both quantitative and qualitative aspects of the expression pattern of CXCL9 or FLJ20174 nucleic acid sequences, including activation or inactivation of CXCL9 or FLJ20174 gene expression.

Diagnostic methods for the detection of CXCL9 or FLJ20174 nucleic acid sequences can involve, for example, contacting and incubating nucleic acids obtained from a biological sample with one or more labeled nucleic acid reagents under conditions favorable for the specific binding of these reagents to their complementary sequences within the CXCL9 or FLJ20174 nucleic acid sequences. After incubation, all unbound nucleic acids are removed, and the presence of nucleic acids that have hybridized, if any such molecules exist, is then detected. Using such a detection scheme, the nucleic acid from the biological sample of interest can be immobilized, for example, to a solid support such as a membrane, or a plastic surface such as that on a microtiter plate or polystyrene beads. Detection of the remaining, bound, labeled CXCL9 or FLJ20174 nucleic acid reagents is accomplished using standard techniques well-known to those in the art. The CXCL9 or FLJ20174 nucleic acid sequences to which the nucleic acid reagents have bound can be compared to the annealing pattern of a normal sample in order to determine whether CXCL9 or FLJ20174 is present in the biological sample tested.

Alternatively, CXCL9 or FLJ20174 nucleic acid sequences can be detected diagnostically in biological samples using nucleic acid amplification techniques, for example, Polymerase Chain Reaction (PCR; the experimental embodiment set forth in Mullis, 1987, U.S. Pat. No. 4,683,202) or Reverse Transcriptase Polymerase Chain Reaction (RT-PCR; CITE), followed by the detection of the amplified molecules using techniques well known to those of skill in the art. The resulting amplified sequences can be compared to those same sequences amplified from a normal sample to determine whether CXCL9 or FLJ20174 is upregulated in the biological sample. In one embodiment of such a detection scheme, a cDNA molecule is synthesized from an RNA molecule of the marker of interest (e.g., by reverse transcription of the RNA molecule into cDNA). A sequence within the cDNA is then used as the template for a nucleic acid amplification reaction, such as a PCR amplification reaction, or the like. The nucleic acid reagents used as synthesis initiation reagents (e.g., primers) in the reverse transcription and nucleic acid amplification steps of this method can be chosen by those of skill in the art from the nucleic acid sequences of CXCL9 or FLJ20174. The amplified product can be detected by using radioactively or non-radioactively labeled nucleotides. Alternatively, enough amplified product may be synthesized such that the product may be visualized by standard ethidium bromide staining or by utilizing any other suitable nucleic acid staining method.

In addition, it is possible to perform such CXCL9 or FLJ20174 expression assays “in situ,” i.e., directly upon tissue sections (fixed and/or frozen) of tissue obtained from biopsies or resections, such that no nucleic acid purification is necessary. Nucleic acid reagents such as those described above may be used as probes and/or primers for such in situ procedures (see, for example, Nuovo, G. J., 1992, “PCR In Situ Hybridization: Protocols And Applications”, Raven Press, NY; OTHER CITE). Alternatively, if a sufficient quantity of the biological sample or cells can be obtained, standard Northern blot analysis can be performed to determine the level of mRNA expression of the CXCL9 or FLJ20174 gene.

Other labeling methods, such as in fluorescent in situ hybridization (FISH), use a fluorescent moiety. FISH involves the incorporation of a substance such as biotinylated UTP into a nucleic acid by means of a nick-translation reaction occurring as the nucleic acid probe is being synthesized. The nucleic acid probe is then denatured and allowed to hybridize, under appropriate conditions, with denatured DNA such as chromosomal DNA. In this way the site of hybridization can be detected by a fluorescently labeled avidin (FITC-avidin) which binds to the biotin on the biotinylated UTP. The signal may be increased by one or more rounds of amplification to identify the unique molecule on the chromatid. It is also possible to use different types of fluorescent labels to look at the positioning of several different probes at the same time and to order their position with respect to each other and to the centromere.

Detection methods useful in the present invention therefore frequently involve the use of isolated nucleic acid sequences. In certain embodiments of the present invention, an isolated nucleic acid molecule of the invention comprises a nucleic acid molecule which has a nucleotide sequence complementary to the nucleotide sequence of a nucleic acid encoded by the gene sequences of CXCL9 or FLJ20174 or to the nucleotide sequence of a nucleic acid encoding a protein which corresponds to the amino acid sequence of the CXCL9 or FLJ20174 gene products. In other embodiments, a nucleic acid molecule of the invention can comprise only a portion of the CXCL9 or FLJ20174 nucleic acid sequences or nucleic acid fragments that encode a polypeptide corresponding to a portion of the CXCL9 or FLJ20174 gene products. Such nucleic acids can be used, for example, as a probe or primer. The probe/primer typically is used as one or more substantially purified oligonucleotides. Such oligonucleotide include nucleic acids comprising 10, 11, 12, 13, 14, 15, 16, 17, 18, 19, 20, 21, 22, 23, 24, 25, 26, 27, 28, 29, 30 or more contiguous nucleotides of SEQ ID NO: 1, SEQ ID NO:3 or SEQ ID NO:4. Alternatively, the oligonucleotide can be substantially homologous to all or a portion of SEQ ID NO: 1, SEQ ID NO:3 or SEQ ID NO:4. Such substantially homologous oligonucleotides comprise a nucleic acid sequence that hybridizes under stringent conditions to at least about 7, preferably about 15, more preferably about 20, 25, 30, 40, 50, 75, 100, 125, 150, 175, 200, 250, 300, 350, or 400 or more consecutive nucleotides of a nucleic acid of the invention.

As used herein, the term “hybridizes under stringent conditions” is intended to describe conditions for hybridization and washing under which nucleotide sequences at least 75% (80%, 85%, preferably 90%) identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). A preferred, non-limiting example of stringent hybridization conditions for specifically binding (annealing) two single-stranded DNA each of which is at least about 100 bases in length and/or for annealing a single-stranded DNA and a single-stranded RNA each of which is at least about 100 bases in length, are hybridization in 6× sodium chloride/sodium citrate (SSC) at about 45° C., followed by one or more washes in 0.2×SSC, 0.1% SDS at 50-65° C. Further preferred hybridization conditions are taught in Lockhart, et al., Nature Biotechnology, Volume 14, 1996 August: 1675-1680; Breslauer, et al., Proc. Natl. Acad. Sci. USA, Volume 83, 1986 June: 3746-3750; Van Ness, et al., Nucleic Acids Research, Volume 19, No. 19, 1991 September: 5143-5151; McGraw, et al., BioTechniques, Volume 8, No. 6 1990: 674-678; and Milner, et al., Nature Biotechnology, Volume 15, 1997 June: 537-541, all expressly incorporated by reference.

Further, naturally-occurring allelic variations of those sequences can exist in a population, including splice-variants, insertions, deletions and point mutations in the CXCL9 and FLJ20174 nucleic acid sequence that result in the expression of an altered gene product. Such variations can be oncogenic or benign in nature. Methods for detecting and determining the effects of such allelic variations are well-known to those of skill in the art.

Detection of CXCL9 or FLJ20174 Gene Products:

The gene products of the CXCL9 and FLJ20174 gene sequences include proteins comprising all or some portion of the amino acid sequences of SEQ ID NO:2, SEQ ID NO:5 and SEQ ID NO:6.

One way to detect the amount of CXCL9 or FLJ20174 gene product in a biological sample is to use an antibody that is specifically reactive with the marker of interest. As used herein, the term “specifically reactive” means an antibody or other ligand that binds specifically with the marker protein, and does not substantially cross-react with any antigen other than then the marker protein. These antibodies may be directed to CXCL9 or FLJ20174 gene products, and may be used as diagnostics and prognostics of breast or ovarian cancer, as described herein. Such methods may be used to detect and quantify an altered pattern of CXCL9 or FLJ20174 gene product synthesis, expression or post-translational modification in a biological sample relative to a normal control, indicating the presence of breast or ovarian cancer, as well as to detect quantitatively or qualitatively the presence of CXCL9 or FLJ20174 gene products.

In one embodiment, expression of the CXCL9 or FLJ20174 gene is assessed using an antibody (e.g. a radio-labeled, chromophore-labeled, fluorophore-labeled, or enzyme-labeled antibody), an antibody derivative (e.g. an antibody conjugated with a substrate or with the protein or ligand of a protein-ligand pair {e.g. biotin-streptavidin}), or an antibody fragment (e.g. a single-chain antibody, an isolated antibody hypervariable domain, etc.) which binds specifically with a protein, encoded by the marker gene, such as the protein encoded by the open reading frame corresponding to the marker gene or such a protein which has undergone all or a portion of its normal post-translational modification.

Immunoassays such as immunofluorescence assays, radioimmunoassays (RIA), immunoblotting, and enzyme linked immunosorbent assays (ELISA) can also be readily adapted to detect CXCL9 or FLJ20174. Both polyclonal and monoclonal antibodies can be used in these assays. For example, immunofluorescence techniques employing a fluorescently labeled antibody coupled with light microscopic, flow cytometric, or fluorimetric detection can be used to detect CXCL9 or FLJ20174 gene products. An example of an ELISA method effective for the detection of CXCL9 or FLJ20174 is as follows: (1) bind the antibody to a substrate; (2) contact the bound antibody with a fluid or tissue sample containing the antigen; (3) contact the above with a secondary antibody bound to a detectable moiety (e.g., horseradish peroxidase enzyme or alkaline phosphatase enzyme); (4) contact the above with the substrate for the enzyme; (5) contact the above with a color reagent; and (6) observe color change. (CITE).

The antibodies useful in the present disclosure may also be employed histologically, as in immunofluorescence or immunoelectron microscopy, for in situ detection of CXCL9 or FLJ20174 gene products. In situ detection may be accomplished by removing a histological specimen from a subject, and applying thereto a labeled antibody of the present disclosure. The antibody is preferably applied by overlaying the labeled antibody onto a biological sample. Through the use of such a procedure, it is possible to determine not only the presence of the CXCL9 or FLJ20174 gene product, but also its distribution in the examined tissue sample. Those of ordinary skill will readily appreciate that any of a wide variety of histological methods (such as staining procedures) can be modified in order to achieve such in situ detection. Immunoassays for CXCL9 or FLJ20174 gene products will typically comprise incubating a biological sample, such as a biological fluid, a tissue extract, freshly harvested cells, or lysates of cells, that have been incubated in cell culture, in the presence of a detectably labeled antibody capable of identifying CXCL9 or FLJ20174 gene products, and detecting the bound antibody by any of a number of techniques well-known to those of skill in the art.

The biological sample may be brought in contact with and immobilized onto a solid phase support or carrier such as nitrocellulose, or other solid support that is capable of immobilizing cells, cell particles, or soluble proteins. The support may then be washed with suitable buffers followed by treatment with the detectably labeled CXCL9 or FLJ20174 gene product-specific antibody. The solid phase support may then be washed with the buffer a second time to remove unbound antibody. The amount of bound label on solid support may then be detected by conventional means. By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present disclosure. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

One of the ways in which the CXCL9 or FLJ20174 gene product-specific antibody can be detectably labeled is by linking the same to an enzyme and using it in an enzyme immunoassay (EIA) (Voller, A. et al., 1978, J. Clin. Pathol. 31, 507-520; Butler, J. E., 1981, Meth. Enzymol. 73, 482-523; Maggio, E. (ed.), 1980, Enzyme Immunoassay, CRC Press, Boca Raton, Fla.,; Ishikawa, E. et al., (eds.), 1981, Enzyme Immunoassay, Kgaku Shoin, Tokyo). The enzyme which is bound to the antibody will react with an appropriate substrate, preferably a chromogenic substrate, in such a manner as to produce a chemical moiety that can be detected, for example, by spectrophotometric, fluorimetric or visual means. Enzymes that can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, glucose oxidase plus peroxidase, beta-galactosidase, ribonuclease, urease, catalase, glucose-6-phosphate dehydrogenase, glucoamylase acetylcholinesterase peroxidase, beta-glucuronidase, beta-D-glucosidase, galactose oxidase plus peroxidase and acid phosphatase. The detection can be accomplished by colorimetric methods that employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays well known to those of skill in the art. For example, by radioactively labeling the antibodies or antibody fragments, it is possible to detect CXCL9 or FLJ20174 gene products through the use of a radioimmunoassay (RIA) (see, for example, Weintraub, B., Principles of Radioimmunoassays, Seventh Training Course on Radioligand Assay Techniques, The Endocrine Society, March, 1986). The radioactive isotope can be detected by such means as the use of a gamma counter, a scintillation counter, autoradiography and the like. Appropriate isotope labels include, but are not limited to: ⁹⁹Tc, ¹⁴C, ¹³¹I, ¹²⁵I, ³H, ³²P and ³⁵S. It is also possible to label the antibody with a fluorescent compound. When the fluorescently labeled antibody is exposed to light of the proper wave length, its presence can be detected due to fluorescence. Among the most commonly used fluorescent labeling compounds are fluorescein and its derivatives, (such as fluorescein isothiocyanate) rhodamine and its derivatives, phycoerythrin, phycocyanin, allophycocyanin, o-phthaldehyde, fluorescamine, auramine, dansyl, umbelliferone, luciferia, 2,3-dihydrophthalazinediones, horseradish peroxidase, alkaline phosphatase, lysozyme, and glycose-6-phosphate dehydrogenase. The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester. Likewise, a bioluminescent compound may be used to label the antibody of the present disclosure. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase, and aequorin.

Gene products of the present invention can also be detected and characterized by other methods known to those of skill in the art including one-dimensional and two-dimensional gel electrophoresis. Post-translational modification of the gene products can be assayed and detected using methods known to those of skill in the art.

Compositions, Kits and Methods for the Detection and Assessment of Breast or Ovarian Cancer

Because certain compositions, kits, and methods of the invention rely on detection of a difference in expression pattern, or specifically the expression level, of CXCL9 or FLJ20174 nucleic acids or gene products, it is preferable that the detection method used be one having a minimum detection limit sufficient to detect any significant variation in the expression pattern of the gene product being detected.

In certain embodiments, the compositions, kits and methods of the present invention are used to detect an up-regulation of the expression level of CXCL9 or FLJ20174 nucleic acid or gene product. In certain embodiments, the expression of CXCL9 or FJL20174 nucleic acid or gene product in a biological sample is preferably up-regulated at least two-fold, three-fold, four-fold or five-fold, and most preferably at least 10-fold relative to normal breast or ovarian tissues obtained from a panel of one or more subjects. In particular, where a comparison between cancerous and normal tissues of an individual subject is desired, the up-regulation of expression in the cancerous tissue is measured relative to the expression levels of one or more healthy tissues of the same type obtained from the same subject.

When the compositions, kits, and methods of the invention are used for characterizing one or more of the stage, grade, histological type, and benign/malignant nature of breast or ovarian cancer in a subject, a positive predictive value (PPV) of greater than about 10% can be obtained for the general population (preferably coupled with an assay specificity greater than 80%). Preferably, compositions, kits and methods of the invention are selected such that the characterization produces a positive result in at least about 20%, and preferably at least about 40%, 60%, or 80%, and more preferably in substantially all subjects afflicted with a breast or ovarian cancer of the corresponding stage, grade, histological type, or benign/malignant nature.

It is recognized that the compositions, kits, and methods of the invention will be of particular utility to patients having an enhanced risk of developing breast or ovarian cancer and their medical advisors. Patients recognized as having an enhanced risk of developing breast or ovarian cancer include, for example, patients having a familial history of breast or ovarian cancer, patients identified as having a mutant oncogene (i.e. at least one allele), and patients determined through any other established medical criteria to be at risk for cancer or other malignancy.

The level of expression of a marker gene in normal (i.e. non-cancerous) human breast or ovarian tissue can be assessed in a variety of ways. In one embodiment, this normal level of expression is assessed by assessing the level of expression of the marker gene in a portion of breast or ovarian cells which appears to be non-cancerous and by comparing this normal level of expression with the level of expression in a portion of the breast or ovarian cells which is suspected of being cancerous. For example, the normal level of expression of a marker gene may be assessed using a non-affected portion of the breast or ovary and this normal level of expression may be compared with the level of expression of the same marker gene in an affected portion of the breast or ovary. Alternately, and particularly as further information becomes available as a result of routine performance of the methods described herein, population-average values for normal expression of the marker genes of the invention may be used. In other embodiments, the ‘normal’ level of expression of a marker gene may be determined by assessing expression of the marker gene in a sample from a subject obtained from a non-cancer-afflicted subject, from a sample from a subject obtained from a subject before the suspected onset of breast or ovarian cancer in the subject, from archived patient samples, and the like.

The invention includes compositions, kits, and methods for assessing the presence of breast or ovarian cancer cells in a sample (e.g. an archived tissue sample or a sample obtained from a patient), and thereby diagnosing the presence of breast or ovarian cancer in a subject. These compositions, kits, and methods are substantially the same as those described above, except that, where necessary, the compositions, kits, and methods are adapted for use with samples other than recently-obtained patient samples. For example, when the sample to be used is a paraffinized, archived human tissue sample, it can be necessary to adjust the ratio of compounds in the compositions of the invention, in the kits of the invention, or the methods used to assess levels of marker gene expression in the sample. Such methods are well known in the art and within the skill of the ordinary artisan.

The invention includes a kit for assessing the presence of breast or ovarian cancer cells (e.g. in a sample such as biological sample from a subject). The kit comprises a plurality of reagents, each of which is capable of binding specifically with a nucleic acid or polypeptide encoded by a marker gene of the invention. Suitable reagents for specifically binding with a polypeptide encoded by a marker gene of the invention include antibodies, antibody derivatives, antibody fragments, and the like. Suitable reagents for specifically binding with a nucleic acid (e.g. a genomic DNA, an mRNA, a spliced mRNA, a cDNA, or the like) include complementary nucleic acids. For example, the nucleic acid reagents may include oligonucleotides (labeled or non-labeled) fixed to a substrate, labeled oligonucleotides not bound with a substrate, pairs of PCR primers, molecular beacon probes, and the like.

The kit of the invention may optionally comprise additional components useful for performing the methods of the invention. By way of example, the kit may comprise fluids (e.g. SSC buffer) suitable for specifically binding complementary nucleic acids or for binding an antibody with a protein with which it specifically binds, one or more sample compartments, an instructional material which describes performance of a method of the invention, a sample of normal breast or ovarian cells, a sample of breast or ovarian cancer cells, and the like.

Methods and kits of the present invention can be used, for example, to monitor the progression of breast or ovarian cancer in a subject. A sample from a subject is obtained at a first point in time and a sample is also obtained at a subsequent point in time. The expression pattern of CXCL9 or FLJ20174, or both, is determined for both samples and compared. The comparison allows for the monitoring of the progression of breast or ovarian cancer in a subject. For example, the first sample can be obtained prior to chemotherapy or other treatment and a subsequent sample obtained after the therapy or treatment. The methods and kits therefore allow for a determination of any changes in the progression or status of the disease after treatment.

Methods and kits of the present invention can also be used to assess a test compound's capacity to act as an effector of breast or ovarian cancer in vivo, i.e. in a subject, or in vitro. The method of assessment involves determining the expression pattern of CXCL9 or FLJ20174 nucleic acids or gene product in a sample before and after the tissue sample is exposed to one or more test compounds. A change in the expression pattern of the sample after treatment with the test compound indicates that the test compound is an effector of breast or ovarian cancer in a subject.

Such methods and kits are useful for the assessment of therapeutic agents to be potentially used in the treatment of a subject having breast or ovarian cancer. One or more therapeutic agents can be exposed to cancerous tissue samples from the patient and the effect of the therapeutic agents on the cancer can be assessed. In this manner, it is possible to screen several different treatment options in order to determine the optimal treatment for a specific subject.

Further, these methods and kits are also useful for the assessment of potential carcinogens. Test compounds suspected of having carcinogenic potential can be screened by exposing them to a normal tissue sample from a subject and determining if exposure to the test compound results in a change in CXCL9 or FLJ20174 nucleic acids or gene product expression. Such changes in expression indicate that the compound has breast or ovarian cell carcinogenic potential.

Screening Assays for Compounds that Modulate the Activity of CXCL9 or FLJ20174 Gene Product:

The following assays are designed to identify compounds that bind to a CXCL9 or FLJ20174 gene product, intracellular proteins or portions of proteins that interact with a CXCL9 or FLJ20174 gene product, compounds that interfere with the interaction of a CXCL9 or FLJ20174 gene product with intracellular proteins, and compounds that modulate the activity of the CXCL9 or FLJ20174 gene (i.e., modulate the level of CXCL9 or FLJ20174 gene expression and/or modulate the level of CXCL9 or FLJ20174 gene product activity). Assays may additionally be utilized that identify compounds that bind to CXCL9 or FLJ20174 gene regulatory sequences (e.g., promoter sequences), and that may modulate the level of CXCL9 or FLJ20174 gene expression. Compounds may include, but are not limited to, small organic or inorganic molecules, peptides, phosphopeptides, antibodies and the like. Not to be limited by theory, CXCL9 is believed to be an extracellular protein and FLJ20174 is believed to be located on the cell surface. Compounds that can interact with CXCL9 and FLJ20174 therefore include, but are not limited to, extracellular proteins or other ligands that affect the function of the CXCL9 or FLJ20174 proteins. Ligands that interact with CXCL9 or FLJ20174 can include compounds ordinarily found in the tissues of the subject as well as therapeutic compounds introduced to the tissue as a means of treating breast or ovarian cancer.

Antibodies, or fragments of antibodies, such as those described below, may be used to screen potentially therapeutic compounds in vitro to determine their effects on CXCL9 or FLJ20174 gene expression and CXCL9 or FLJ20174 protein production. The compounds that have beneficial effects on breast or ovarian cancer can be identified, and a therapeutically effective dose determined.

Therapeutic Agents and Methods of Treatment of Breast or Ovarian Cancer

The invention provides for both prophylactic and therapeutic methods of treating a subject suffering from or at risk for breast or ovarian cancer.

Cancers and pre-cancerous conditions that are characterized by increased expression levels or biological activity of CXCL9 or FLJ20174 can be treated with therapeutics that antagonize (i.e., reduce or inhibit) that expression or activity. Therapeutics that antagonize expression or activity may be administered in a therapeutic or prophylactic manner. Therapeutics that can be utilized include, but are not limited to: (i) antibodies to FLJ26174 or CXCL9 gene products; (ii) antisense nucleic acid and nucleic acids that are “dysfunctional” (e.g., due to a heterologous insertion within the coding sequences of a CXCL9 or FLJ20174 nucleic acids sequence) that are utilized to “knockout” expression of the CXCL9 or FLJ20174 gene by homologous recombination (see, e.g., Capecchi, 1989. Science 244: 1288-1292); (iii) RNA interference (RNAi) oligonucleotides that silence or reduce expression of the CXCL9 and FLJ20174 gene products or (iv) modulators (i.e., inhibitors, agonists and antagonists, including natural ligands, artificial ligands or peptide mimetics of the CXCL9 or FLJ20174 gene products or antibodies specific to the CXCL9 or FLJ20174 gene products) that alter the interaction between the CXCL9 or FJL20174 gene product and one or more of its binding partners.

Cancers and pre-cancerous conditions that are characterized by decreased expression levels or biological activity of CXCL9 or FLJ20174 can be treated with therapeutics that increase (e.g., are agonists to) expression or activity. Therapeutics that upregulate expression activity may be administered in a therapeutic or prophylactic manner.

Increased or decreased expression levels or biological activity of FLJ20174 and CXCL9 can be readily detected by quantifying FLJ20174 or CXCL9 gene product, FLJ20174 or CXCL9 RNA, or both, in a sample from a subject (e.g., from biopsy tissue) and assaying it in vitro for RNA or gene product levels, structure and/or activity of the expressed proteins and the like using detection methods such as those described above.

In one aspect, the invention provides a method for preventing, in a subject, a breast or ovarian cancer, by administering to the subject an agent that modulates expression of an FLJ20174 or CXCL9 nucleic acid or gene product, or that modulates at least one FLJ20174 or CXCL9 gene product activity. Subjects at risk for a disease that is caused or contributed to by differential FLJ20174 or CXCL9 expression or activity can be identified by, for example, any or a combination of diagnostic or prognostic assays as described herein. Administration of a prophylactic agent can occur prior to the manifestation of symptoms characteristic of the FLJ20174 or CXCL9 aberrancy, such that a disease or disorder is prevented or, alternatively, delayed in its progression. Depending upon the type of FLJ20174 or CXCL9 aberrancy, for example, a FLJ20174 or CXCL9 agonist or FLJ20174 or CXCL9 antagonist agent can be used for treating the subject. The appropriate agent can be determined based on screening assays described herein.

Another aspect of the invention pertains to methods of modulating FLJ20174 or CXCL9 expression or activity for therapeutic purposes. The modulatory method of the invention involves contacting a cell with an agent that modulates the expression of the FLJ20174 or CXCL9 nucleic acids or gene products or that modulates one or more of the activities of the FLJ20174 or CXCL9 gene product. An agent that modulates FLJ20174 or CXCL9 activity can be an agent as described herein, such as a nucleic acid or a protein, a naturally-occurring cognate ligand of a FLJ20174 or CXCL9 protein, a peptide, peptidomimetic, or other small molecule. In one embodiment, the agent stimulates one or more activities of the FLJ20174 or CXCL9 gene products. Examples of such stimulatory agents include (i) active FLJ20174 or CXCL9 protein and (ii) a nucleic acid molecule encoding FLJ20174 or CXCL9 that has been introduced into the cell. In another embodiment, the agent inhibits one or more FLJ20174 or CXCL9 protein activity. Examples of such inhibitory agents include antisense FLJ20174 or CXCL9 nucleic acid molecules, FLJ20174 or CXCL9 RNA interference oligonucleotides, anti-FLJ20174 or anti-CXCL9 antibodies and FLJ20174 or CXCL9 ligands. These modulatory methods can be performed in vitro (e.g., by culturing the cell with the agent) or, alternatively, in vivo (e.g., by administering the agent to a subject). As such, the invention provides methods of treating an individual afflicted with a cancer or pre-cancerous condition characterized by aberrant expression or activity of a FLJ20174 or CXCL9 protein or nucleic acid molecule. In one embodiment, the method involves administering an agent, or combination of agents that modulates or inhibits FLJ20174 or CXCL9 expression or activity. In another embodiment, the method involves administering a FLJ20174 or CXCL9 protein or nucleic acid molecule as therapy to compensate for reduced or aberrant FLJ20174 or CXCL9 expression or activity.

Antibodies of the invention, including polyclonal, monoclonal, humanized and fully human antibodies, may be used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target.

A therapeutically effective amount of an antibody of the invention relates generally to the amount needed to achieve a therapeutic objective. Not to be limited by theory, the therapeutic effect can be caused by a binding interaction between the antibody and its target antigen that interferes with the functioning of the target, and in other cases, the therapeutic effect can be caused by a physiological response to the formation of the antibody-target complex. The amount required to be administered will depend on the binding affinity of the antibody for its specific antigen, the effect of the binding on the target molecule and on the rate at which an administered antibody is depleted from the subject of tissue to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week. To produce an enhanced therapeutic effect, antibodies may be conjugated to a radioactive agent such as technetium-99, a chemotherapeutic agent such as doxorubicin or paclitaxel, a biological toxin such as ricin, or some other agent that is generally harmful to cells. Conjugation with a selective antibody allows these relatively non-selective agents to be preferentially delivered to cancer cells, concentrating their effects and sparing non-cancerous tissue. Such conjugated antibodies may also be used as imaging agents to provide enhanced detection of cancerous tissue.

Alternatively, nucleic acid compositions such as antisense oligonucleotides or RNA interference oligonucleotides may be used to reduce or inhibit the expression of FLJ20174 or CXCL9 mRNA and gene product.

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Pharmaceutical Compositions

The nucleic acids, gene products, antibodies and ligands of the present invention may also be used in methods of treating breast or ovarian cancer. Compounds that have been determined to modulate the expression of CXCL9 or FLJ20174 nucleic acids, gene products or both can be administered to a subject to prevent the development of precancerous or cancerous tissue, to inhibit the conversion of precancerous tissue to cancerous tissue, or to ameliorate in whole or in part the symptoms or progression of breast or ovarian cancer.

Where clinical applications are contemplated, it will be necessary to prepare pharmaceutical compositions in a form appropriate for the intended application. Pharmaceutical compositions disclosed herein can be administered to a subject using a variety of routes of administration and dosage forms well known to those of skill in the art. As used herein, “pharmaceutical compositions” of the present invention include compositions comprising agents that have been found to modulate the expression pattern of CXCL9 or FLJ20174 nucleic acid, gene products or both. “Pharmaceutical compositions” of the present invention also include compositions of antibodies or conjugated antibodies that have been found to bind preferentially to cancer cells. Generally, this will entail preparing compositions that are essentially free of pyrogens, as well as other impurities that could be harmful to cells, humans, or animals.

Pharmaceutical compositions of the present invention may be administered through a number of different routes, including oral, peroral, enteral, pulmonary, rectal, nasal, vaginal, lingual, direct injection, intravenous, intraarterial, intracardial, intradermal, intramuscular, intraperitoneal, intracutaneous, intraocular, intranasal, intrapleural, intrathecal, intratumor, intrauterine, orthotopic, and subcutaneous administration. Pharmaceutical compositions of the present invention can also be suitable for systemic administration to the subject, including parenteral, topical, buccal, sublingual, transdermal, gavage, and oral administration. Pharmaceutical compositions of the present invention may also be administered parenterally, i.e. subcutaneously, intramuscularly, or intravenously. Compositions can also be delivered to a tissue or organism in a variety of different compositions, including tablets, pills, capsules, powders, aerosols, suppositories, skin patches, parenterals, and oral liquids, including oil-aqueous suspensions, solutions, and emulsions. Also contemplated is the administration of pharmaceutical compositions in single or multiple dosage regimens, as well as by using compositions that involve sustained release (long acting) formulations and devices.

In addition to other routes of administration, agents that modulate the expression of CXCL9 and FLJ20174 can be presented to the tissue of interest by direct infusion or by using expression vectors or constructs, including but not limited to genetically modified cells or tissues, viral vectors, or infusion of genetic material, which can then be incorporated by cells or in the tissue of a host organism. Alternatively, a pharmaceutical composition of the present invention can be applied topically or injected directly into a tissue of interest (e.g., injected as a bolus within a tumor or intercutaneous or subcutaneous site, applied to all or a portion of the surface of the skin, dropped onto the surface of the eye, etc.). Additionally, a pharmaceutical composition of the present invention can be incorporated into biodegradable polymers that allow for the sustained release of the compound. The polymers can be implanted in the vicinity of where drug delivery is desired, for example, at the site of a tumor, or implanted so that the compound that modulates the expression pattern of CXCL9 or FLJ20174 nucleic acid, gene product or both is slowly released systemically. The biodegradable polymers and their use are described, for example, in Brem et al., J. Neurosurg. 74:441-446, 1991, which is incorporated herein by reference.

The phrase “pharmaceutically or pharmacologically acceptable” refers to molecular entities and compositions that do not produce adverse, allergic, or other untoward reactions when administered to an organism, animal, or human. As used herein, “pharmaceutically acceptable carrier” includes any and all solvents, dispersion media, coatings, antibacterial and antifingal agents, isotonic and absorption delaying agents and the like. The use of such media and agents for pharmaceutically active substances is well known in the art. Except insofar as any conventional media or agent is incompatible with the vectors or cells of the present disclosure, its use in therapeutic compositions is contemplated. Supplementary active ingredients also can be incorporated into the compositions.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

The expression level of CXCL9 was tested in various normal and cancerous tissue samples using RT-PCR. The nucleotide sequences of the primers used to amplify a 300 base pair cDNA fragment of CXCL9 are as follows:

| | |
|---|---|
| ACACTCACTACAGGGACCAG, | (SEQ ID NO: 7) |
| AND | |
| ATAAGGATCTCGGTGGCTAT. | (SEQ ID NO: 8) |

Human breast cancers and matching normal breast tissues were obtained with informed consent from patients at UT Southwestern Medical Center and ILSBio, LLC (Bethesda, Md.). Human ovarian cancers and normal ovarian tissues were obtained with informed consent from patients at UT Southwestern Medical Center. Normal human tissue RNA was obtained from BD Biosciences (Clontech). Human tissues were frozen in liquid nitrogen within 30 minutes of harvesting and stored at −80° C. Total RNA was isolated from tissues using Trizol (Invitrogen).

Total RNA from each tissue sample was treated with DNaseI, Amplification Grade (Invitrogen) according to manufacturer's recommendations. One microgram of DNaseI-treated RNA was denatured at 70° C. for 5 minutes with 0.1 U oligo dT primers (Amersham Biosciences) and reverse transcribed with 100 U Superscript™II reverse transcriptase (Invitrogen) in 1× First-strand buffer [250 mM Tris-HCl (pH8.3), 375 mM KCl, 15 mM MgCl2], 0.05 μg/μl BSA, 1 mM DTT, 40 U RNaseOUT™, and 0.5 mM dNTPs, at 42° C. for 1.5-2 hours. The reaction mixture was diluted 1:10 with H₂O and 5 μl were used for PCR amplification. The twenty base-pair PCR primers identified as SEQ ID NO: 7 and SEQ ID NO: 8 were chosen to amplify approximately 200 bp of the target gene using Primer3 software (Rozen, S. & Skaletsky, H. (2000) Methods Mol Biol 132, 365-86) and synthesized by Qiagen or Illumina, Inc. (San Diego, Calif.). PCR reactions were set up as follows: 0.3 μM PCR primers were combined with 5 μl diluted cDNA in a final volume of 50 μl consisting of 1.6 mM MgCl2, 20 mM Tris-HCl (pH 8.4), 50 mM KCl, 0.2 mM dNTPs, and 1.25 U Taq DNA Polymerase (Invitrogen). Samples were denatured in a T1 Thermocycler (Whatman, Biometra) at 94° C. for 2 minutes, incubated at 94° C. for 30 seconds, 55° C. for 30 seconds and 72° C. for 30 seconds for 25 to 33 cycles, and extended at 72° C. for 5 minutes. Samples were resolved in 2% agarose and products were visualized with EtBr under UV light.

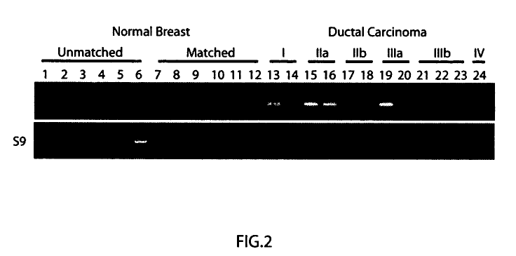

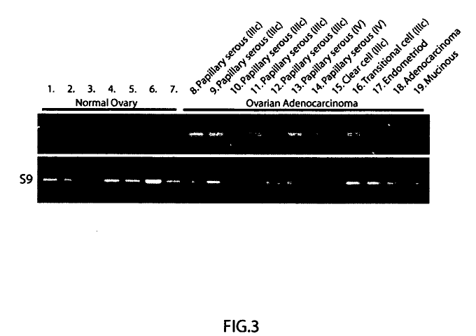

The results of the tissue panels are shown in FIGS. 1-3. The results of these and other studies indicate that the expression levels of CXCL9 are much lower in all normal tissues examined (n=18) compared to breast cancer (n=4) and ovarian cancer (n=4) (FIG. 1). CXCL9 is over-expressed in approximately 58% of the ductal breast carcinomas (n=12) examined compared to normal breast tissue (n=12) (FIG. 2). CXCL9 was found to be similarly over-expressed in 75% of the ovarian adenocarcinoma samples (n=12) examined compared to normal ovarian tissue (n=9) (FIG. 3).

### EXAMPLE 2

The expression level of FLJ20174 was tested in various normal and cancerous tissue samples using RT-PCR. The nucleotide sequences of the primers used to amplify a 248 bp cDNA fragment common to the “wild-type” and “splice variant” isoforms of FLJ20174 are as follows:

| | | |
|---|---|---|
| Primer 1: | CTTCAGGAGTCCTCCACAGC | (SEQ ID NO: 9) |
| Primer 2: | CAGACACAGAGCTGCCAGAC | (SEQ ID NO: 10) |

The nucleotide sequences of the primers used to amplify a 300 bp cDNA fragment specific to the “splice variant” and a 373 bp cDNA fragment specific to the “wild type” isoform of FLJ20174 are as follows:

| | | |
|---|---|---|
| Primer 1: | AGGAGAGCGACTTCGACACC | (SEQ ID NO: 11) |
| Primer 2: | GCTCTCCGATGGAGGATGTC | (SEQ ID NO: 12) |

Human breast cancers and matching normal breast tissues were obtained with informed consent from patients at UT Southwestern Medical Center and ILSBio, LLC (Bethesda, Md.). Human ovarian cancers and normal ovarian tissues were obtained with informed consent from patients at UT Southwestern Medical Center. Normal human tissue RNA was obtained from BD Biosciences (Clontech). Human tissues were frozen in liquid nitrogen within 30 minutes of harvesting and stored at −80° C. Total RNA was isolated from tissues using Trizol (Invitrogen).

Total RNA was treated with DNaseI, Amplification Grade (Invitrogen) according to manufacturer's recommendations. One microgram of DNaseI-treated RNA was denatured at 70° C. for 5 minutes with 0.1 U oligo dT primers (Amersham Biosciences) and reverse transcribed with 100 U Superscript™II reverse transcriptase (Invitrogen) in 1× First-strand buffer [250 mM Tris-HCl (pH8.3), 375 mM KCl, 15 mM MgCl2], 0.05 μg/μl BSA, 1 mM DTT, 40 U RNaseOUT™, and 0.5 mM dNTPs, at 42° C. for 1.5-2 hours. The reaction mixture was diluted 1:10 with H₂O and 5 μl were used for PCR amplification. Twenty base-pair PCR primers pairs (SEQ ID NOS. 9-12) were chosen to amplify 248 or 373 bp of the target gene using Primer3 software (Rozen, S. & Skaletsky, H. (2000) Methods Mol Biol 132, 365-86) and synthesized by Qiagen or Illumina, Inc. (San Diego, Calif.). PCR reactions were set up as follows: 0.3 μM PCR primers were combined with 5 μl diluted cDNA in a final volume of 50 μl consisting of 1.6 mM MgCl2, 20 mM Tris-HCl (pH 8.4), 50 mM KCl, 0.2 mM dNTPs, and 1.25 U Taq DNA Polymerase (Invitrogen). Samples were denatured in a T1 Thermocycler (Whatman, Biometra) at 94° C. for 2 minutes, incubated at 94° C. for 30 seconds, 55° C. for 30 seconds and 72° C. for 30 seconds for 25 to 33 cycles, and extended at 72° C. for 5 minutes. Samples were resolved in 2% agarose and products were visualized with EtBr under UV light.

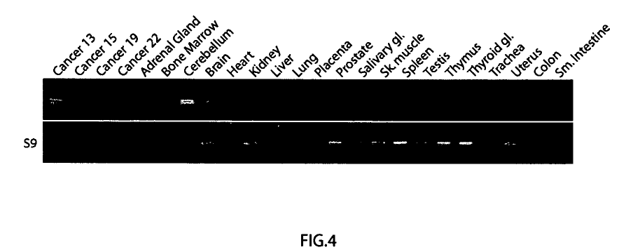

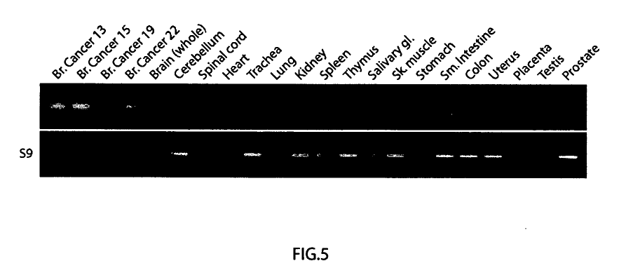

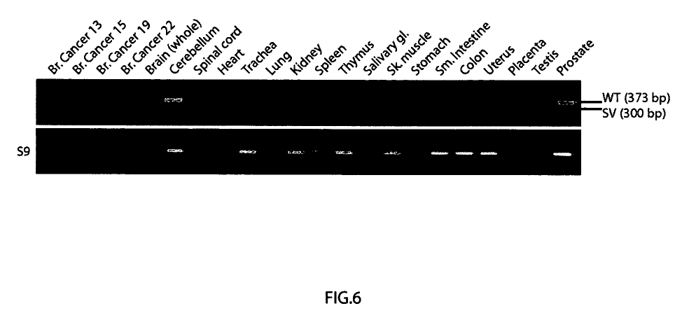

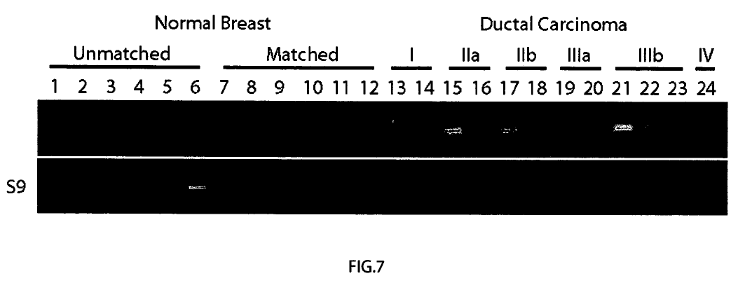

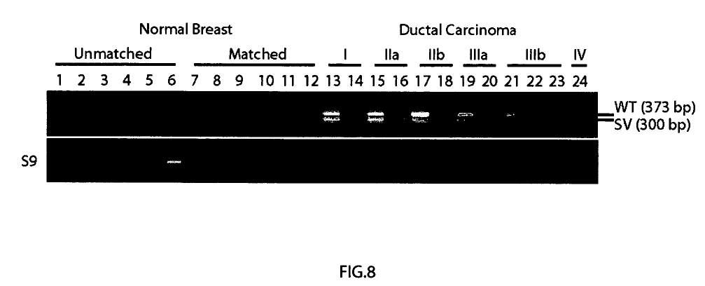

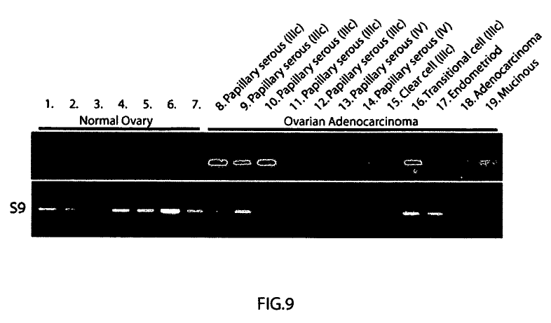

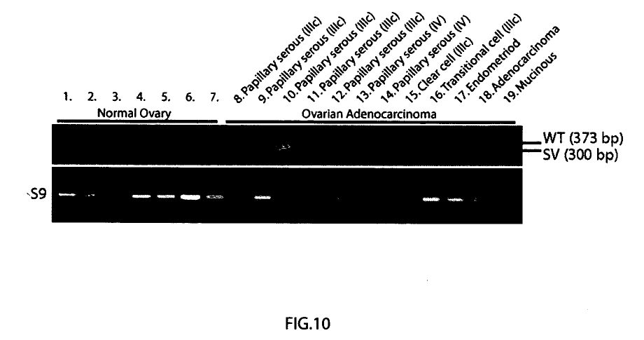

The results of the expression analysis by RT-PCR are shown in FIGS. 4-10. The results from these and other experiments indicate that FLJ20174 is expressed in several normal human tissues (n=18), however the level of expression is generally lower than that detected in breast cancers (n=4) (FIG. 4 and FIG. 5). Importantly, the “splice variant” isoform of FLJ20174 appeared to be expressed at higher levels in the breast cancers compared to other normal human tissues whereas the “wild type” isoform was detected at similar levels (FIG. 6). This may indicate that the “splice variant” isoform is a more cancer-specific target. FLJ20174 was overexpressed in 75% of the ductal breast carcinomas (n=12) examined compared to normal breast tissue (n=12) (FIG. 7). The “wild type” and “splice variant” isoforms displayed identical patterns of expression in the breast cancers (FIG. 8). FLJ20174 was also found to be overexpressed in approximately 58% of the ovarian cancers examined (n=12) relative to normal ovary tissue (n=9) (FIG. 9). Similar to that observed in breast cancer, the “wild type” and “splice variant” isoforms exhibited similar patterns of expression in the ovarian cancers (FIG. 10).

### EXAMPLE 3

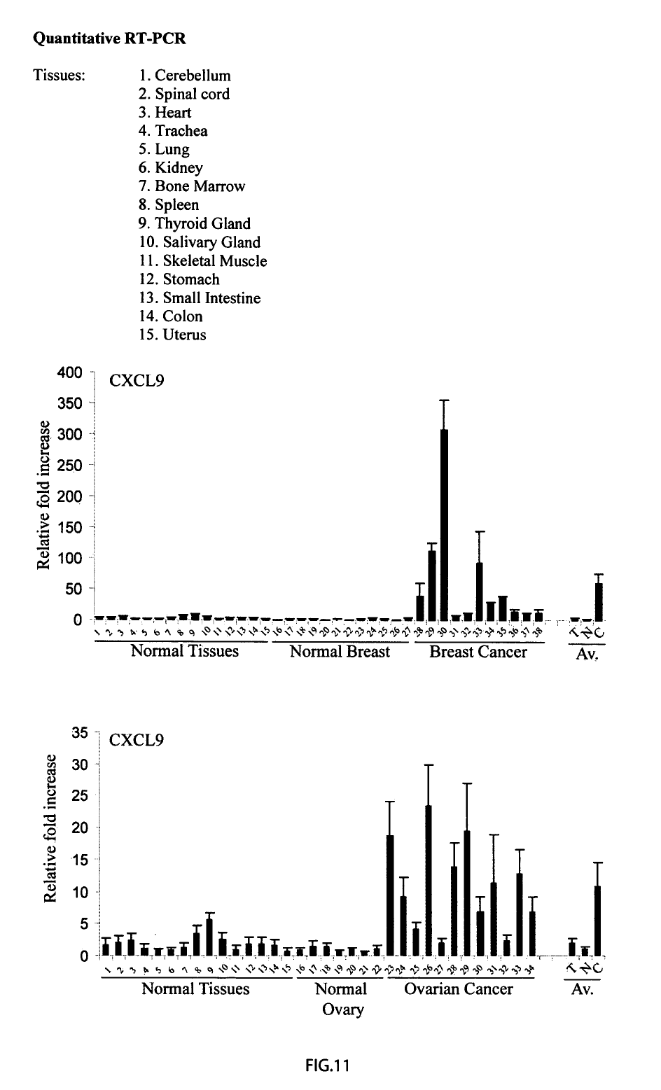

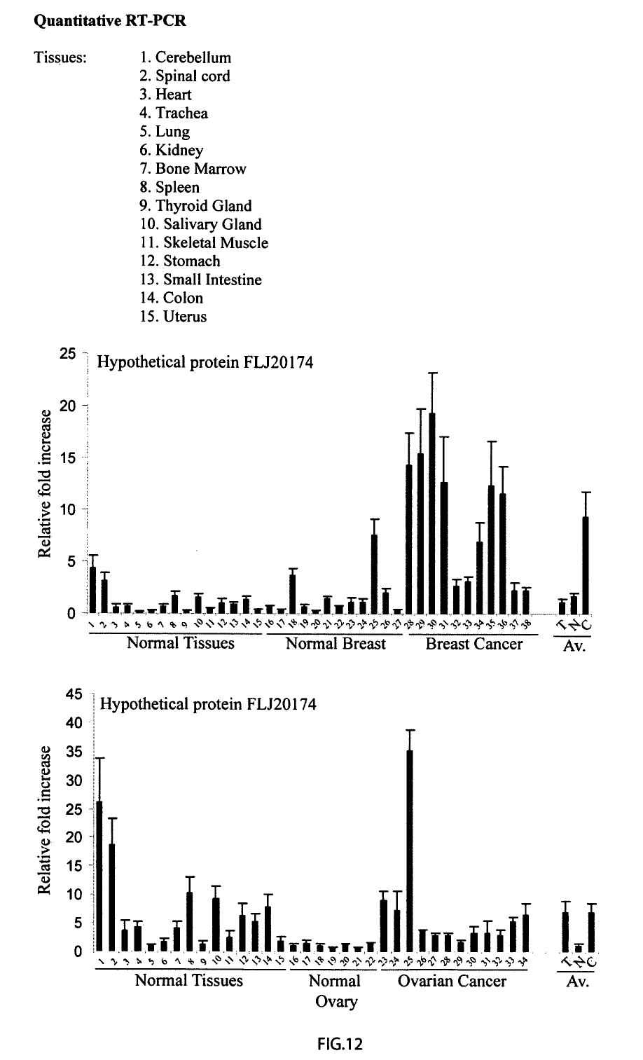

FIGS. 11 and 12 show the results of quantitative RT-PCR performed to detect the level of expression of CXCL9 or FLJ20174, respectively, in normal tissues and breast and ovarian cancers.

Total RNA was treated with DNaseI, Amplification Grade (Invitrogen) according to manufacturer's recommendations. One microgram of DNaseI-treated RNA was denatured at 70° C. for 5 minutes with 0.1 U oligo dT primers (Amersham Biosciences) and reverse transcribed with 100 U Superscript™II reverse transcriptase (Invitrogen) in 1× First-strand buffer [250 mM Tris-HCl (pH8.3), 375 mM KCl, 15 mM MgCl2], 0.05 μg/μl BSA, 1 mM DTT, 40 U RNaseOUT™, and 0.5 mM dNTPs, at 42° C. for 1.5-2 hours. 5 μL of cDNA (diluted 1:10) was used in a final reaction volume of 30 μL consisting of 1× DyNAmo SYBR Green qPCR mix (MJ Research). Products were amplified using the DNA Engine Opticon® 2 Continuous Fluorescence Detection System (MJ Research). Samples were denatured at 94° C. for 10 minutes, incubated at 94° C. for 15 seconds, 55° C. for 15 seconds and 72° C. for 15 seconds for 40 cycles, and extended at 72° C. for 5 minutes. Products of the primer pairs of SEQ ID NOS: 7 and 8 and products of primer pairs SEQ ID NOS: 9 and 10 were each subjected to a melting curve to ensure that dimers were not produced. Relative levels of gene expression were determined using the comparative Ct (ΔΔCt) method using primers specific for S9 ribosomal RNA as a control.

The results for CXCL9 indicate that the average level in breast cancer was approximately 47-fold higher than the average level in normal breast tissues (FIG. 11). Compared to normal ovarian tissues, the average CXCL9 level detected in ovarian adenocarcinoma was approximately 10-fold higher (FIG. 11). Compared to the average level of CXCL9 in normal human tissues, an average of approximately 21-fold and approximately 6-fold increases in expression levels were observed in breast cancer and ovarian cancer, respectively (FIG. 11).

The results for FLJ20174 indicate that the average level in breast cancer was ˜5.5-fold higher than the average level in normal breast tissues (FIG. 12). Compared to normal ovarian tissues, the average FLJ20174 level detected in ovarian adenocarcinoma was approximately 6.6-fold higher (FIG. 12). Comparing the average expression level of FLJ20174 in a variety of non-breast normal human tissues (e.g. liver, lung, heart, etc.) to the average expression level in breast cancer tissues, a approximately 8.1-fold increase in expression was observed in breast cancer cells over that of normal non-breast tissues (FIG. 12). However the average FLJ20174 expression levels in non-ovarian normal human tissues was approximately equivalent to the expression level found in ovarian adenocarcinomas.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents that are chemically or physiologically related may be substituted for the agents described herein while the same or similar results would be achieved. All such similar substitutes and modifications apparent to those skilled in the art are deemed to be within the spirit, scope and concept of the invention as defined by the appended claims.

1. A method of diagnosing breast cancer in a subject, the method comprising comparing the expression pattern of CXCL9 or FLJ20174 nucleic acid or gene product in a sample from a subject with the expression pattern of CXCL9 or FLJ20174 nucleic acid or gene product in one or more samples from one or more non-cancerous tissues, wherein a difference in the expression pattern of CXCL9 or FLJ20174 in the samples is indicative of breast or ovarian cancer in the subject.

2. The method of claim 1 wherein the sample from the subject is derived from the same tissue type as the one or more samples from a non-cancerous tissue.

3. The method of claim 2 wherein the one or more samples from a non-cancerous tissue are also derived from the subject.

4. The method of claim 1 wherein the difference in the expression pattern is an up-regulation of the expression level of CXCL9 or FLJ20174 nucleic acid.

5. The method of claim 2, wherein the difference in the expression pattern is an up-regulated of at least two fold over the level of expression of CXCL9 or FLJ20174 nucleic acid in the one or more non-cancerous tissue samples.

6. The method of claim 1, wherein the sample comprises cells obtained from the subject.

7. The method of claim 4 wherein the cells are obtained from breast or ovarian tissue.

8. The method of claim 1, wherein the sample comprises serum, nipple aspirate or ductal fluid obtained from the subject.

9. The method of claim 1 wherein the expression pattern of CXCL9 or FLJ20174 is determined by detecting the presence in the sample of a nucleic acid comprising 30 or more contiguous nucleotides of SEQ ID NO: 1, SEQ ID NO:3 or SEQ ID NO:4.

10. The method of claim 7, wherein the transcribed polynucleotide is an mRNA or hnRNA.

11. The method of claim 7, wherein the transcribed polynucleotide is a cDNA.

12. The method of claim 7, wherein the step of detecting further comprises amplifying the transcribed polynucleotide.

13. The method of claim 1, wherein the expression pattern CXCL9 or FLJ20174 in the samples is assessed by detecting the presence in the samples of a transcribed polynucleotide which specifically binds with SEQ ID NO: 1, SEQ ID NO:3 or SEQ ID NO:4 or specifically binds with a portion of said transcribed polynucleotides, under stringent hybridization conditions.

14. The method of claim 1 wherein the difference in the expression pattern is an up-regulation of the expression level of CXCL9 or FLJ20174 gene product.

15. The method of claim 1, wherein the expression pattern of CXCL9 or FLJ20174 gene product is determined by detecting the presence in the samples of a protein, polypeptide or protein fragment of SEQ ID NO:2, SEQ ID NO:5 or SEQ ID NO:6.

16. The method of claim 13, wherein the presence of said protein, polypeptide or protein fragment is detected using a reagent which specifically binds with said protein, polypeptide or protein fragment.

17. The method of claim 14, wherein the reagent is selected from the group consisting of an antibody, an antibody derivative, and an antibody fragment.

18. A method of assessing the prognosis of a breast or ovarian cancer subject, the method comprising comparing the expression pattern of CXCL9 or FLJ20174 in a sample from a subject with the expression pattern of CXCL9 or FLJ20174 in samples from one or more subjects suffering from a known type of breast or ovarian cancer and determining the prognosis based on the comparison.

19. The method of claim 16 wherein the expression pattern is assessed by determining the level of expression.

20. The method of claim 16 wherein the expression pattern is assessed by comparing expression patterns of CXCL9 or FLJ20174 in different tissue samples from the same subject.

21. The method of claim 16 wherein the expression pattern is assessed by comparing the level of post-translational modification of CXCL9 or FLJ20174.

22. The method of claim 16, wherein the sample comprises cells obtained from the subject.

23. The method of claim 20 wherein the cells are obtained from breast or ovarian tissue.

24. The method of claim 16, wherein the sample comprises serum, nipple aspirate or ductal fluid obtained from the subject.

25. The method of claim 16, wherein the level of expression of CXCL9 or FLJ20174 is determined by detecting the presence in the sample of a nucleic acid comprising 10 or more contiguous nucleotides of SEQ ID NO: 1, SEQ ID NO:3 or SEQ ID NO:4.

26. The method of claim 23, wherein the level of expression of said marker genes in the sample is assessed by detecting the presence in the sample of a transcribed polynucleotide encoded by SEQ ID NO: 1, SEQ ID NO:3 or SEQ ID NO:4 or a portion of said transcribed polynucleotides.

27. The method of claim 23, wherein the transcribed polynucleotide is an mRNA or hnRNA.

28. The method of claim 23, wherein the transcribed polynucleotide is a cDNA.

29. The method of claim 23, wherein the step of detecting further comprises amplifying the transcribed polynucleotide.

30. The method of claim 16, wherein the level of expression of CXCL9 or FLJ20174 in the sample is assessed by detecting the presence in the sample of a transcribed polynucleotide which specifically binds with SEQ ID NO: 1, SEQ ID NO:3 or SEQ ID NO:4 or specifically binds with a portion of said transcribed polynucleotides, under stringent hybridization conditions.

31. The method of claim 16, wherein the level of expression of CXCL9 or FLJ20174 is determined by detecting the presence in the sample of a protein, polypeptide or protein fragment of SEQ ID NO:2, SEQ ID NO:5 or SEQ ID NO:6.

32. The method of claim 29, wherein the presence of said protein, polypeptide or protein fragment is detected using a reagent which specifically binds with said protein, polypeptide or protein fragment.

33. The method of claim 30, wherein the reagent is selected from the group consisting of an antibody, an antibody derivative, and an antibody fragment

34. A method for monitoring the progression of breast or ovarian cancer in a subject, the method comprising:
a. determining in a sample from a subject at a first point in time the expression pattern of CXCL9 or FLJ20174;
b. repeating step a) at a subsequent point in time; and
c. comparing the expression pattern of the CXCL9 or FLJ20174 determined in steps a) and b), and therefrom monitoring the progression of breast or ovarian cancer.

35. The method of claim 32 wherein the expression pattern is assessed by determining the level of expression.

36. The method of claim 32 wherein the expression pattern is assessed by comparing expression patterns of CXCL9 or FLJ20174 in different tissue samples from the same subject.

37. The method of claim 32 wherein the expression pattern is assessed by comparing the level of post-translational modification of CXCL9 or FLJ20174.

38. The method of claim 32 wherein the cells are obtained from breast or ovarian tissue.

39. The method of claim 33, wherein the sample comprises serum, nipple aspirate or ductal fluid obtained from the subject.

40. The method of claim 32, wherein between the first point in time and the subsequent point in time, the subject has undergone surgery to remove breast or ovarian tissue.

41. A method of assessing a test compound as an effector of breast or ovarian cancer in a subject, the method comprising
a. determining the expression pattern of CXCL9 or FLJ20174 in a first tissue sample from a subject;
b. exposing one or more second tissue samples from the same subject to one or more test compounds;
c. determining the expression pattern of CXCL9 or FLJ20174 in the one or more tissue samples that have been exposed to one or more test compounds; and
d. comparing the expression pattern of CXCL9 or FLJ20174 in the first and second tissue samples,
wherein a change in the expression pattern of CXCL9 or FLJ20174 in the second tissue sample versus the first tissue sample indicates that the test compound is an effector of breast or ovarian cancer in a subject.

42. The method of claim 39 wherein the first tissue sample exhibits an abnormal CXCL9 or FLJ20174 expression pattern and wherein the test compound is a therapeutic agent.

43. The method of claim 39 wherein the test compound is assessed to determine the breast or ovarian cell carcinogenic potential of the test compound.

44. A kit for diagnosis of breast or ovarian cancer, the kit comprising reagents for assessing the expression pattern of CXCL9 or FLJ20174 in a sample from a subject.

45. The kit of claim 42 wherein the kit comprises a reagent which specifically binds with a transcribed polynucleotide of SEQ ID NO: 1, SEQ ID NO:3 or SEQ ID NO:4.

46. The kit of claim 42 wherein the kit comprises a reagent which specifically binds to a protein, polypeptide or protein fragment of SEQ ID NO:2, SEQ ID NO:5 or SEQ ID NO:6.

47. A kit for assessing the suitability of one or more test compounds for treating breast or ovarian cancer in a subject, the kit comprising: a) one or more test compounds; and b) a reagent for assessing the expression pattern of CXCL9 or FLJ20174.

48. The kit of claim 43 wherein the kit comprises a probe which specifically binds with a transcribed polynucleotide of SEQ ID NO: 1, SEQ ID NO:3 or SEQ ID NO:4.

49. The kit of claim 43 wherein the kit comprises a probe which specifically binds to a protein, polypeptide or protein fragment of SEQ ID NO:2, SEQ ID NO:5 or SEQ ID NO:6.

50. A therapeutic agent for the treatment of breast or ovarian cancer comprising an agent that specifically binds to a CXCL9 or FLJ20174 nucleic acid or gene product.

51. The therapeutic agent of claim 50 wherein the agent specifically binds to a protein, polypeptide or protein fragment of SEQ ID NO:2, SEQ ID NO:5 or SEQ ID NO:6.

52. The therapeutic agent of claim 51 wherein the agent is an antibody, an antibody derivative, and an antibody fragment.

53. The therapeutic agent of claim 50 wherein the agent specifically binds with a polynucleotide of SEQ ID NO: 1, SEQ ID NO:3 or SEQ ID NO:4.

54. The therapeutic agent of claim 53 wherein the agent is an antisense nucleic acid.

55. The therapeutic agent of claim 53 wherein the agent is an RNA interference oligonucleotide.


SEQUENCE LISTING (12 sequences)

SEQ ID NO: 1 - DNA, 2545 bp, Homo sapiens
atccaatacaggagtgacttggaactccattctatcactatgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataagagaccacttcaccaataagtattctgtgttaaaaatgttctattttaattataccgctatcattccaaaggaggatggcatataatacaaaggcttattaatttgactagaaaatttaaaacattactctgaaattgtaactaaagttagaaagttgattttaagaatccaaacgttaagaattgttaaaggctatgattgtctttgttcttctaccacccaccagttgaatttcatcatgcttaaggccatgattttagcaatacccatgtctacacagatgttcacccaaccacatcccactcacaacagctgcctggaagagcagccctaggcttccacgtactgcagcctccagagagtatctgaggcacatgtcagcaagtcctaagcctgttagcatgctggtgagccaagcagtttgaaattgagctggacctcaccaagctgctgtggccatcaacctctgtatttgaatcagcctacaggcctcacacacaatgtgtctgagagattcatgctgattgttattgggtatcaccactggagatcaccagtgtgtggctttcagagcctcctttctggctttggaagccatgtgattccatcttgcccgctcaggctgaccactttatttctttttgttcccctttgcttcattcaagtcagctcttctccatcctaccacaatgcagtgcctttcttctctccagtgcacctgtcatatgctctgatttatctgagtcaactcctttctcatcttgtccccaacaccccacagaagtgctttcttctcccaattcatcctcactcagtccagcttagttcaagtcctgcctcttaaataaacctttttggacacacaaattatcttaaaactcctgtttcacttggttcagtaccacatgggtgaacactcaatggttaactaattcttgggtgtttatcctatctctccaaccagattgtcagctccttgagggcaagagccacagtatatttccctgtttcttccacagtgcctaataatactgtggaactaggttttaataattttttaattgatgttgttatgggcaggatggcaaccagaccattgtctcagagcaggtgctggctctttcctggctactccatgttggctagcctctggtaacctcttacttattatcttcaggacactcactacagggaccagggatgatgcaacatccttgtctttttatgacaggatgtttgctcagcttctccaacaataagaagcacgtggtaaaacacttgcggatattctggactgtttttaaaaaatatacagtttaccgaaaatcatataatcttacaatgaaaaggactttatagatcagccagtgaccaaccttttcccaaccatacaaaaattccttttcccgaaggaaaagggctttctcaataagcctcagctttctaagatctaacaagatagccaccgagatccttatcgaaactcattttaggcaaatatgagttttattgtccgtttacttgtttcagagtttgtattgtgattatcaattaccacaccatctcccatgaagaaagggaacggtgaagtactaagcgctagaggaagcagccaagtcggttagtggaagcatgattggtgcccagttagcctctgcaggatgtggaaacctccttccaggggaggttcagtgaattgtgtaggagaggttgtctgtggccagaatttaaacctatactcactttcccaaattgaatcactgctcacactgctgatgatttagagtgctgtccggtggagatcccacccgaacgtcttatctaatcatgaaactccctagttccttcatgtaacttccctgaaaaatctaagtgtttcataaatttgagagtctgtgacccacttaccttgcatctcacaggtagacagtatataactaacaaccaaagactacatattgtcactgacacacacgttataatcatttatcatatatatacatacatgcatacactctcaaagcaaataatttttcacttcaaaacagtattgacttgtataccttgtaatttgaaatattttctttgttaaaatagaatggtatcaataaatagaccattaatcag

SEQ ID NO: 2 - DNA, 4669 bp, Homo sapiens
tttttattattgctgttattgaggttgagggagaagagatcggtctaaattctggctgggtaagtggggggattctcggcgatgagaaacgggggacttagaagccggaggaaaatcagcagccccacatctccacttctccagtccgccctactctccacccgtgacctccagtggagaccccaggcggcagcatcagtatttgatcggcccttcgtcagcacgctgccagccctggccggctgggttcgccaggcatcacccgctcggctctgaagcggacgcctggccctgcaccgggctttggaaggaccctctctgcgctcgccccctccccagggtggctccgctttcgagcccgggcgcggcgcccaccatgcgcggctgcctgcggctcgcgctgctctgcgcgctgccctggctcctgctggcggcgtcgcccgggcacccggcgaaatcccccaggcagcccccggcaccgcgccgcgaccccttcgacgctgccaggggcgccgatttcgatcatgtctacagcggggtggtgaacctcagcaccgagaacatctactctttcaactacaccagccagcccgaccaggtgacagccgtgagggtgtatgtgaacagttcctctgagaatctcaactacccggtccttgttgtggttcgccagcagaaagaggtgctgtcctggcaggttcctctgctcttccaaggactataccagaggagctacaattatcaagaagtgagccgcaccttatgtccctcagaagcaaccaatgagacgggacccttgcagcaactgatatttgtagatgtcgcatccatggcacccctgggtgctcagtacaaactgctagttaccaagctgaagcacttccagctccggacaaatgttgcctttcactttactgccagcccctctcaacctcagtattttctatacaagtttcccaaagacgtggactcagttatcattaaagtggtgtctgaaatggcttatccatgttctgttgtctcagtccagaatatcatgtgcccggtgtatgatctcgaccacaatgtggaatttaatggtgtctatcagtccatgaccaagaaagctgccatcacgctacagaagaaggattttccaggcgagcagttcttcgtggtatttgtgataaagcctgaagattatgcctgtggaggatctttcttcatccaggaaaaggaaaaccagacctggaatctacagcgaaaaaagaaccttgaagtgaccattgtcccttccattaaagaatctgtttatgtgaaatccagtcttttcagtgtcttcatcttcctgtccttctacttgggatgccttcttgttgggtttgttcattatctgaggtttcagagaaaatccattgatggaagctttgggtccaatgatggctctggaaatatggtggcatctcatcccattgctgccagcacacccgaagggagcaattatgggacaatagatgagtcaagctccagtcctggaaggcagatgtcctcctccgatggtgggccaccgggccagtcagacacagacagctccgtggaggagagcgacttcgacaccatgccagacattgagagtgataaaaacatcatccggaccaagatgttcctttacctgtcagatttgtccaggaaggaccggagaattgtcagcaaaaaatataaaatttatttttggaacatcatcaccattgctgtgttttacgcgctgcccgtgatccagctggtcattacctatcagacagtggtaaatgtcactggcaaccaggacatctgttactacaacttcctctgtgctcaccccttgggcgtcctgagtgccttcaacaacattctcagcaatctgggccacgtgcttctgggcttcctcttcctgctgatagtcttgcgccgcgacatcctccatcggagagccctggaagccaaggacatctttgctgtggagtacgggattcccaaacactttggtctcttctacgctatgggcattgcattgatgatggaaggggtgctcagtgcttgctaccatgtctgccctaattattccaacttccgattcgacacctccttcatgtacatgatcgctggcctgtgcatgctgaagctctatcagacccgccacccagacatcaatgccagcgcctactctgcctatgcctcctttgctgtggtcatcatggtcaccgtccttggagtggtgtttggaaaaaatgacgtatggttctgggtcatcttctctgcaatccacgttctggcctcgctagccctcagcacccagatatattatatgggtcgtttcaagatagatttgggaattttccggcgggctgccatggtgttctacacagactgtatccagcagtgtagccgacctctatatatggatagaatggtgttgctggttgtggggaatctggttaactggtccttcgccctctttggattgatataccgccccagggactttgcttcctacatgctgggcatcttcatctgtaaccttttgctgtacctggccttttacatcatcatgaagctccgcagctctgaaaaggtcctcccagtcccgctcttctgcatcgtggccaccgctgtgatgtgggctgccgccctatattttttcttccagaatctcagcagctgggagggaactccggccgaatcccgggagaagaaccgcgagtgcattctgctggatttcttcgatgaccatgacatctggcacttcctctctgctactgctctgtttttctcattcttggttttgttaactttggatgatgaccttgatgtggttcggagagaccagatccctgtcttctgaacctccaacattaagagaggggagggagcgatcaatcttggtgctgtttcacaaaaattacagtgaccacagcaaagtaaccactgccagatgctccactcaccctctgtagagccaactctgcattcacacaggaaggagaggggctgcgggagatttaaacctgcaagaaaggaggcagaaggggagccatgttttgaggacagacgcaaacctgaggagctgagaaacacttgctccttccatctgcagctttgggagtgcaacagggataggcactgcatccaagtcaactcaccatcttggggtccctcccaccctcacggagacttgccagcaatggcagaatgctgctgcacacttccctccagttgtcaccctgcccagaaaggccagcagcttggacttcctgcccagaaactgtgttggcccccttcacacctctgcaacacctgctgctccagcaagaggatgtgattctttagaatatggcggggaggtgaccccaggccctgccctactgggatagatgttttaatggcaccagctagtcacctcccagaagaaactctgtacatttcccccaggtttctgatgccatcagaagggctcaggagtggggtttgtcacacattcctcttaacaagtaactgtcactgggaccgagtcctgggtgcttacatattccttcgtgtcttcatctcactgacctgtgtggacctcatcactctgactctgccttcttggaaaggccctgtcactccacagatgtctggccagcttcaaggcagaaggaaaaacaggaaaagctcttttaacagcagcaggaacaagagaaatgactaaccatactaaaagactggtaacagcagcagcagccagacaggcctcaccttaaggacttgggctgccagagcaaattcagcagagcttatttggcctcccattcacacagctcagttctgtgcccacatcacctttggggaagaaatcagcattctaatcagggacactacttcaggagtcctccacagcgagtccgtcatctgtcactttatgtagatcagggttctagacttcttccctgaggttctcagaagcagctctcaggatgaacgtattgtcctcttcccctcttcttgcaaagtgcacagctaatctaatgttgtctctcggttgcacctgacattctctccccagtaaggtgttggcaagctcagcatctgggttccactctcacactgtctggcagctctgtgtctgagaagttctacattgaccaggcccccttgttgcctggagtatgacgtaatcagaaaatagacgtataaatgtgcacatgcgtatgtatttgcttgtgaaattaaagtcacctcttgcctctgctttcctgatcattcgttagagaaatggatcaggcatttttttaaattattattctttctctaaactatttgcattgtgttcaaaaacccattttagaagtttgaacagcaagcttttcctgattttaaaaacacaaagttgctttcaatgaaatattttgtgatttttttaaagtccccaaatgtgtacttagccttctgttattccttattctttaagcagtgttggcttccattgaccatatgaaggccaccaattaaatggttgtgttaatccaacatgtaaaaaactttttggcagggcacagtggctcacgcctgtaatcccaaagtgctggaatttacaggtaggaccaccacacttggtccacttacttataataaacattgatttggtcgttaaaaaaaaaaaaaaaaaaaaaaa

SEQ ID NO: 3 - DNA, 4595 bp, Homo sapiens
tttttattattgctgttattgaggttgagggagaagagatcggtctaaattctggctgggtaagtggggggattctcggcgatgagaaacgggggacttagaagccggaggaaaatcagcagccccacatctccacttctccagtccgccctactctccacccgtgacctccagtggagaccccaggcggcagcatcagtatttgatcggcccttcgtcagcacgctgccagccctggccggctgggttcgccaggcatcacccgctcggctctgaagcggacgcctggccctgcaccgggctttggaaggaccctctctgcgctcgccccctccccagggtggctccgctttcgagcccgggcgcggcgcccaccatgcgcggctgcctgcggctcgcgctgctctgcgcgctgccctggctcctgctggcggcgtcgcccgggcacccggcgaaatcccccaggcagcccccggcaccgcgccgcgaccccttcgacgctgccaggggcgccgatttcgatcatgtctacagcggggtggtgaacctcagcaccgagaacatctactctttcaactacaccagccagcccgaccaggtgacagccgtgagggtgtatgtgaacagttcctctgagaatctcaactacccggtccttgttgtggttcgccagcagaaagaggtgctgtcctggcaggttcctctgctcttccaaggactataccagaggagctacaattatcaagaagtgagccgcaccttatgtccctcagaagcaaccaatgagacgggacccttgcagcaactgatatttgtagatgtcgcatccatggcacccctgggtgctcagtacaaactgctagttaccaagctgaagcacttccagctccggacaaatgttgcctttcactttactgccagcccctctcaacctcagtattttctatacaagtttcccaaagacgtggactcagttatcattaaagtggtgtctgaaatggcttatccatgttctgttgtctcagtccagaatatcatgtgcccggtgtatgatctcgaccacaatgtggaatttaatggtgtctatcagtccatgaccaagaaagctgccatcacgctacagaagaaggattttccaggcgagcagttcttcgtggtatttgtgataaagcctgaagattatgcctgtggaggatctttcttcatccaggaaaaggaaaaccagacctggaatctacagcgaaaaaagaaccttgaagtgaccattgtcccttccattaaagaatctgtttatgtgaaatccagtcttttcagtgtcttcatcttcctgtccttctacttgggatgccttcttgttgggtttgttcattatctgaggtttcagagaaaatccattgatggaagctttgggtccaatgatggctctggaaatatggtggcatctcatcccattgctgccagcacacccgaagggagcaattatgggacaatagatgagtcaagctccagtcctggaaggcagatgtcctcctccgatggtgggccaccgggccagtcagacacagacagctccgtggaggagagcgacttcgacaccatgccagacattgagagtgataaaaacatcatccggaccaagatgttcctttacctgtcagatttgtccaggaaggaccggagaattgtcagcaaaaaatataaaatttatttttggaacatcatcaccattgctgtgttttacgcgctgcccgtgatccagctggtcattacctatcagacatgccttcaacaacattctcagcaatctgggccacgtgcttctgggcttcctcttcctgctgatagtcttgcgccgcgacatcctccatcggagagccctggaagccaaggacatctttgctgtggagtacgggattcccaaacactttggtctcttctacgctatgggcattgcattgatgatggaaggggtgctcagtgcttgctaccatgtctgccctaattattccaacttccgattcgacacctccttcatgtacatgatcgctggcctgtgcatgctgaagctctatcagacccgccacccagacatcaatgccagcgcctactctgcctatgcctcctttgctgtggtcatcatggtcaccgtccttggagtggtgtttggaaaaaatgacgtatggttctgggtcatcttctctgcaatccacgttctggcctcgctagccctcagcacccagatatattatatgggtcgtttcaagatagatttgggaattttccggcgggctgccatggtgttctacacagactgtatccagcagtgtagccgacctctatatatggatagaatggtgttgctggttgtggggaatctggttaactggtccttcgccctctttggattgatataccgccccagggactttgcttcctacatgctgggcatcttcatctgtaaccttttgctgtacctggccttttacatcatcatgaagctccgcagctctgaaaaggtcctcccagtcccgctcttctgcatcgtggccaccgctgtgatgtgggctgccgccctatattttttcttccagaatctcagcagctgggagggaactccggccgaatcccgggagaagaaccgcgagtgcattctgctggatttcttcgatgaccatgacatctggcacttcctctctgctactgctctgtttttctcattcttggttttgttaactttggatgatgaccttgatgtggttcggagagaccagatccctgtcttctgaacctccaacattaagagaggggagggagcgatcaatcttggtgctgtttcacaaaaattacagtgaccacagcaaagtaaccactgccagatgctccactcaccctctgtagagccaactctgcattcacacaggaaggagaggggctgcgggagatttaaacctgcaagaaaggaggcagaaggggagccatgttttgaggacagacgcaaacctgaggagctgagaaacacttgctccttccatctgcagctttgggagtgcaacagggataggcactgcatccaagtcaactcaccatcttggggtccctcccaccctcacggagacttgccagcaatggcagaatgctgctgcacacttccctccagttgtcaccctgcccagaaaggccagcagcttggacttcctgcccagaaactgtgttggcccccttcacacctctgcaacacctgctgctccagcaagaggatgtgattctttagaatatggcggggaggtgaccccaggccctgccctactgggatagatgttttaatggcaccagctagtcacctcccagaagaaactctgtacatttcccccaggtttctgatgccatcagaagggctcaggagtggggtttgtcacacattcctcttaacaagtaactgtcactgggaccgagtcctgggtgcttacatattccttcgtgtcttcatctcactgacctgtgtggacctcatcactctgactctgccttcttggaaaggccctgtcactccacagatgtctggccagcttcaaggcagaaggaaaaacaggaaaagctcttttaacagcagcaggaacaagagaaatgactaaccatactaaaagactggtaacagcagcagcagccagacaggcctcaccttaaggacttgggctgccagagcaaattcagcagagcttatttggcctcccattcacacagctcagttctgtgcccacatcacctttggggaagaaatcagcattctaatcagggacactacttcaggagtcctccacagcgagtccgtcatctgtcactttatgtagatcagggttctagacttcttccctgaggttctcagaagcagctctcaggatgaacgtattgtcctcttcccctcttcttgcaaagtgcacagctaatctaatgttgtctctcggttgcacctgacattctctccccagtaaggtgttggcaagctcagcatctgggttccactctcacactgtctggcagctctgtgtctgagaagttctacattgaccaggcccccttgttgcctggagtatgacgtaatcagaaaatagacgtataaatgtgcacatgcgtatgtatttgcttgtgaaattaaagtcacctcttgcctctgctttcctgatcattcgttagagaaatggatcaggcatttttttaaattattattctttctctaaactatttgcattgtgttcaaaaacccattttagaagtttgaacagcaagcttttcctgattttaaaaacacaaagttgctttcaatgaaatattttgtgatttttttaaagtccccaaatgtgtacttagccttctgttattccttattctttaagcagtgttggcttccattgaccatatgaaggccaccaattaaatggttgtgttaatccaacatgtaaaaaactttttggcagggcacagtggctcacgcctgtaatcccaaagtgctggaatttacaggtaggaccaccacacttggtccacttacttataataaacattgatttggtcgttaaaaaaaaaaaaaaaaaaaaaaa

SEQ ID NO: 4 - protein, 82 aa, Homo sapiens
MKKSGVGVGVGTVVRKGRCSCSTNGTHSKDKASSCKATKNGVTCNDSADVKKKWKVSKKKKNGKKHKKKVKVRKSRSRKKTT

SEQ ID NO: 5 - protein, 529 aa, Homo sapiens
MRGCRACAWAASGHAKSRARRDDAARGADDHVYSGVVNSTNYSNYTSDVTAVRVYVNSSSNNYVVVVRKVSWVGYRSYNYVSRTCSATNTGVDVASMAGAYKVTKKHRTNVAHTASSYYKKDVDSVKVVSMAYCSVVSVNMCVYDDHNVNGVYSMTKKAATKKDGVVVKDYACGGSKNTWNRKKNVTVSKSVYVKSSSVSYGCVGVHYRRKSDGSGSNDGSGNMVASHAASTGSNYGTDSSSSGRMSSSDGGGSDTDSSVSDDTMDSDKNRTKMYSDSRKDRRVSKKYKYWNTAVYAVVTYTVVNVTGNDCYYNCAHGVSANNSNGHVGVRRDHRRAAKDAVYGKHGYAMGAMMGVSACYHVCNYSNRDTSMYMAGCMKYTRHDNASAYSAYASAVVMVTVGVVGKNDVWWVSAHVASASTYYMGRKDGRRAAMVYTDCCSRYMDRMVVVGNVNWSAGYRRDASYMGCNYAYMKRSSKVVCVATAVMWAAAYNSSWGTASRKNRCDDDHDWHSATASVTDDDDVVRRDV

SEQ ID NO: 6 - protein, 346 aa, Homo sapiens
MRGCRACAWAASGHAKSRARRDDAARGADDHVYSGVVNSTNYSNYTSDVTAVRVYVNSSSNNYVVVVRKVSWVGYRSYNYVSRTCSATNTGVDVASMAGAYKVTKKHRTNVAHTASSYYKKDVDSVKVVSMAYCSVVSVNMCVYDDHNVNGVYSMTKKAATKKDGVVVKDYACGGSKNTWNRKKNVTVSKSVYVKSSSVSYGCVGVHYRRKSDGSGSNDGSGNMVASHAASTGSNYGTDSSSSGRMSSSDGGGSDTDSSVSDDTMDSDKNRTKMYSDSRKDRRVSKKYKYWNTAVYAVVTYTCHSSGRASGADSARHSSGSGHCCGVRDSTWSRYGHCDDGRGACC

SEQ ID NO: 7 - DNA, 20 bp, Homo sapiens
acactcactacagggaccag

SEQ ID NO: 8 - DNA, 20 bp, Homo sapiens
ataaggatctcggtggctat

SEQ ID NO: 9 - DNA, 20 bp, Homo sapiens
cttcaggagtcctccacagc

SEQ ID NO: 10 - DNA, 20 bp, Homo sapiens
cagacacagagctgccagac

SEQ ID NO: 11 - DNA, 20 bp, Homo sapiens
aggagagcgacttcgacacc

SEQ ID NO: 12 - DNA, 20 bp, Homo sapiens
gctctccgatggaggatgtc